(19) United States
(12) Patent Application Publication
(10) Pub. No.: US 2007/0231256 A1
(43) Pub. Date: Oct. 4, 2007

(54) Compositions and methods for detecting and treating tumors containing acidic areas

(76) Inventor: James E. Summerton, Corvallis, OR (US)

Correspondence Address: Teri G. Andrews, Patent Attomey, 907 NW Raintree Dr., Corvallis, OR, 97330, US

(21) Appl. No.: 11/395,487

(22) Filed: Mar. 30, 2006

(51) Int. Cl. A61K 51/00

(52) U.S. Cl. 424/1.69; 424/185.1

(57) ABSTRACT

Improved compositions have been developed which are selectively sequestered in acidic areas of tumors. When the compositions contain a radioisotope effective to report the presence of the composition, the compositions are useful for detecting tumors. When the compositions contain radioisotopes effective to kill cells, the compositions are useful for treating tumors.

### BACKGROUND OF THE INVENTION

### Field of the Invention

This invention relates to compositions which are selectively sequestered in acidic areas of tumors for the purpose of detecting and killing tumors.

### INTRODUCTION

It has been known for many decades that most or all tumors larger than about 1 millimeter in diameter contain hypoxic/acidic areas. The likely cause of this is that for tumors to grow larger than about 1 mm in diameter they must induce new blood vessels, and such tumor-induced blood vessels, particularly the capillaries, are abnormal, being too widely spaced, torturous in path, and their walls are excessively permeable. As a consequence, cells more than a few tens of microns from such tumor capillaries commonly are chronically hypoxic and the interstitial space surrounding them is acidic, ranging from as low as about pH 6.0 in areas most distant from capillaries, up to about pH 7.0 closer to capillaries, with pH values in the range of about 6.5 to 6.8 being most common. This acidity is probably due in substantial part to the hypoxia causing the tumor cells to shift to glycolytic metabolism, which leads to their producing and excreting lactic acid. While tumor cells at near-normal pH in close proximity to capillaries have high metabolic rates and fast cell division, those tumor cells in hypoxic/acidic areas a greater distance from capillaries have low metabolic rates and divide slowly or not at all. These slow and non-dividing tumor cells are called quiescent.

The quiescent tumor cells in hypoxic/acidic areas of tumors probably constitute the greatest impediment to long-term success with conventional cancer therapies. This is because while conventional cancer therapies (radiation and chemotherapeutics) are fairly effective in killing fast-dividing cells, such as those tumor cells in close proximity to tumor capillaries, most such therapies have been explicitly selected for their ability to spare slow-dividing and non-dividing cells typical of most normal tissues. Therefore, not surprisingly, conventional cancer therapies are also rather ineffective against slow-dividing and non-dividing quiescent tumor cells. As a consequence, tumor treatments typically kill predominantly the fast-dividing cells of a tumor while sparing quiescent cells of that tumor. This initial killing of the fast-dividing tumor cells causes the tumor to go into remission, but after those killed cells have been disposed of by the body's normal cleanup processes, all too often the treatment-resistant quiescent cells in the hypoxic/acidic areas of the tumor slowly regain access to adequate oxygen, nutrients, and waste disposal—allowing them to revert to high metabolic rate and fast cell division. This rejuvenation of the previously quiescent tumor cells commonly leads to the dreaded relapse that kills so many patients.

The hypoxic/acidic properties of tumors have been known for over 75 years and it has long been speculated that such properties might be exploitable for therapy. However, to date it appears the most successful efforts to exploit these properties have focused on the hypoxia. Specifically, substances have been developed which exhibit minimal cytotoxicity in normoxic cells, while exhibiting considerable cytotoxicity in hypoxic cells. One such agent has progressed to the clinical trials stage.

Compared to exploiting the hypoxia in tumors, until quite recently there appears to have been much less success in exploiting the acidity of tumors. One unsuccessful approach was based on the observation that acid pH in tissues acts to sensitize those tissues to thermal damage. However, efforts to exploit this acid-mediated sensitivity of cells gave disappointing results.

Another approach relating to acidity in tumors is based on the fact that the low pH in tumors ionizes weak-base cytotoxic agents and thereby renders them membrane-impermeable, which in turn results in preferential reduction of entry of a number of such weak-base agents into acidic areas of tumors relative to entry of such agents into cells in areas of more normal pH. In this regard, rather than attempting to exploit the low pH in the tumor, efforts were instead focused on raising the pH in the tumors as a means to partially de-ionize and thereby enhance the entry of such weak-base cytotoxic agents into cells of the tumor, and such efforts have met with some success.

### RELATED ART

Still another approach, and the only one which relates to the present invention, relies on the fact that the low pH in the interstitial space of tumors will effect partial de-ionization of weak-acid cytotoxic agents. As a consequence of this lesser degree of ionization of weak-acid agents in acidic areas of tumors, one would expect that such agents should show enhanced entry and hence greater cytotoxicity against cells in the acidic areas of tumors. This expectation has been tested by Kozin et al., wherein they took measures to make tumors in tumor-bearing mice more acidic by established methods (Cancer Research Vol. 61, pages 4740-4743 (2001)). They reported that, as predicted, a reduction of about 0.3 pH units in the tumors coincided with a modest 1.7-fold improvement in tumor growth delay afforded by the weak-acid (pKa 5.8) cytotoxic agent, Chlorambucil (shown in FIG. 1). In the conclusion to their paper they wrote: “To our knowledge, CHL (Chlorambucil) is the only clinical therapeutic that is a weak-acid with the appropriate pKa≦6.5. This study thus provides a rationale for the design of novel, potent drugs exhibiting similar weak-acid properties and for which diffusion contributes to intracellular uptake. As also shown here, the combined use of such compounds with radiation and/or modulators of the pH gradient provide additional opportunities for maximizing the therapeutic response.”

In the aforementioned paper, the authors note that their results with Chlorambucil provide a rationale for designing new weak-acid anti-cancer drugs which may show high efficacy—but no guidance is given on prospective molecular structures for such drugs, nor is any guidance given on what specific properties are desirable, nor is any guidance given on how to go about designing and preparing such weak-acid drugs, nor is any guidance given concerning applications or methods of use of such drugs. In contrast to the above, a number of years ago James Summerton, inventor of the present invention, began the development of novel peptide structures explicitly designed to exploit the pH differential between normal tissues and acidic regions of tumors, with the objective of providing safer and more effective treatments for a broad range of tumors. Early peptide compositions and methods for detection and treatment of tumors, which were developed in the course of that work, are detailed in two pending patent applications submitted by applicant (patent application Ser. Nos. 11/069,849, pending; and 11/069,387, allowed but not yet published or issued). In the present patent application, the inventor describes subsequent improvements and extensions in the design, preparation, properties, applications, and methods of use of novel compositions designed to exploit the acidity of tumors for the purpose of achieving very early detection and safe and effective treatment of a broad range of tumors. Such compositions contain “pH-switches” which at the pH of normal tissues exist predominantly in a form which has little or no affinity for tissues, but when they enter an acidic area of a tumor they switch in substantial part to a form which is sequestered in that tumor.

### SUMMARY OF THE INVENTION

Improved compositions have been developed which are selectively sequestered in acidic areas of tumors. When the compositions contain a radioisotope effective to report the presence of the composition, the compositions are useful for detecting tumors. When the compositions contain radioisotopes effective to kill cells, the compositions are useful for treating tumors.

### BRIEF DESCRIPTION OF THE FIGURES

FIG. 1 shows the weak-acid chemotherapeutic, chlorambucil.

FIG. 2 illustrates the distribution of acidity in tumors.

FIG. 3 illustrates the previously-postulated acid-pairs in peptides.

FIG. 4a shows a non-acid-pairable peptide sequence.

FIG. 4b shows representative end structures for improved pH-switch peptides.

FIG. 5a shows preferred amino acid sequences for improved pH-switch peptides.

FIG. 5b shows axial distribution plots of glutamic acid side chains of the preferred amino acid sequences improved pH-switch peptides.

FIG. 6 is a series of axial distribution plots illustrating the amino acid sequences unsuitable as pH-switches.

FIG. 7a-7d illustrates a series of lipophilic end groups for previous pH-switch peptides, and the pH at mid-point of membrane binding of each.

FIG. 8 is a diagram illustrating the pH-mediated transition between forms.

FIG. 9 shows calculated titration curves as a function of lipophilicities of the acid form.

FIG. 10a illustrates waters H-bonded to a conventional carboxylic acid in its salt form and acid form.

FIG. 10b illustrates waters H-bonded to an internal acid-specific H-bonding carboxylic acid in its salt form and acid form.

FIG. 11a illustrates a structure which forms an internal acid-specific H-bond.

FIG. 11b illustrates a structure which forms non-acid-specific H-bonds.

FIG. 12a shows representative ring structures suitable for advanced pH-switches.

FIG. 12b shows an unacceptable structure for advanced pH-switches.

FIG. 13 is a table demonstrating insulation of a carboxyl from inductive effects as a function of the number of carbons separating the carbonyl from the phenyl ring.

FIG. 14a illustrates an H-bond site which is open to solvent.

FIG. 14b illustrates an H-bond site which is partially shielded from solvent.

FIG. 15a is a table showing an H-bond donor and representative H-bond acceptors suitable for forming low-barrier H-bonds.

FIG. 15b illustrates representative advanced pH-switches with low-barrier H-bonds.

FIGS. 16a and 16b illustrate elements in a structural optimization procedure.

FIG. 17a illustrates an oligomeric onco tool with two advanced pH-switches.

FIG. 17b illustrates an oligomeric onco tool with three advanced pH-switches.

FIG. 18a illustrates a dimeric advanced pH-switch structure.

FIG. 18b shows the n-octanol/buffer partitioning of a dimeric advanced pH-switch structure.

FIG. 19 illustrates onco tools with multiple advanced pH-switches.

FIG. 20 shows representative core structures for advanced pH-switches.

FIG. 21 shows a cargo component for large onco tools.

FIG. 22 shows two cargo components for small onco tools.

FIG. 23 shows the steps in preparation of an onco tool containing an improved pH-switch peptide component.

FIG. 24 shows the steps in preparation of an onco tool containing an improved pH-switch peptide component, plus an advanced pH-switch component.

FIG. 25 shows synthetic routes for preparing onco tools containing a single advanced pH-switch component.

FIG. 26 shows synthetic routes for preparing onco tools containing multiple advanced pH-switch components.

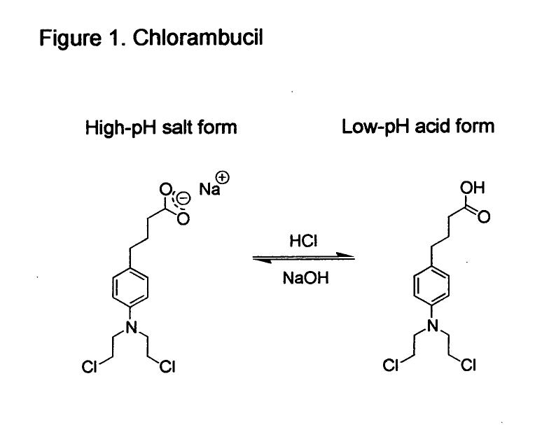

### OVERVIEW OF pH-SWITCH STRATEGY FOR DETECTING AND TREATING TUMORS CONTAINING ACIDIC AREAS

Two key terms used in describing this novel diagnostic and treatment strategy are as follows:

pH-Switch means a structural component which at the pH present in normal tissues (pH 7.4) exists predominantly in an anionic hydrophilic form which: a) has little or no affinity for tissues; and, b) favors rapid excretion from the body through the kidneys. When that pH-switch component enters an acidic area of a tumor it undergoes in substantial part a pH-mediated switch to a non-ionic lipophilic acid form which is sequestered in the tumor by virtue of binding to extra-cellular structures and/or binding to cell surfaces and/or entering into cells.

Onco Tool means a composition that includes one or more pH-switch components and one or more cargo components, where a cargo component is effective to report the presence of the composition and/or is effective to kill cells. A “diagnostic onco tool” is an onco tool that includes at least one cargo component effective to report the presence of the onco tool. A “therapeutic onco tool” is an onco tool that includes at least one cargo component effective to kill cells.

A. Diagnostic Application of pH-Switch Strategy

An ideal diagnostic for tumors should: 1) be effective to detect a wide range of tumor types; 2) be effective to detect even very small tumors; 3) be effective to distinguish between tumor and all normal tissues; 4) provide an adequate signal to noise ratio for decisive detection of tumors which are present; and, 5) provide a suitable signal readily detected outside the body. Conventional tumor diagnostic reagents and methods typically can only detect tumors when the tumor is about 1 centimeter in diameter or larger, and such diagnostics are commonly not brought to bear until after the tumor has caused overt symptoms—by which time the tumor has often metastasized and/or caused irreversible organ damage. Further, because of the great variability between tumor types, and even between tumors similar in type, routine detection of tumors remains difficult and uncertain. In contrast to conventional tumor diagnostic reagents and methods, the inventor believes pH-switch-containing onco tools, as described hereafter, can serve to effectively detect a wide range of tumor types at very early stages of tumor development, and can be effective for readily distinguishing even very small tumors from surrounding normal tissues.

1. Detect a Wide Range of Tumor Types

Much of the difficulty in routine early detection of tumors has been in identifying some property that is unique to tumors while also being common to a wide range of tumor types, and which can be readily exploited for routine, affordable, and non-invasive detection of tumors. In contrast to the wide variety of tumor-associated properties which are currently exploited for detecting many different types of tumors, the acidity in the interstitial space of hypoxic areas of tumors appears to be virtually unique to tumors, while also being common to a very wide range of tumor types. Accordingly, compositions designed to exploit such tumor acidity are expected to be effective for the detection of most, and possibly all tumor types.

2. Detect Even Very Small Tumors

Acidic regions in tumors typically begin to form when the tumors reach a size of about 1 millimeter in diameter. In this regard, it should be noted that a near-microscopic size tumor 1 millimeter in diameter has a volume about a thousand fold smaller than the volume of a tumor 1 centimeter in diameter, and that thousand-fold larger 1 centimeter tumor is about the current minimum detectable size for conventional tumor diagnostics. Thus, in contrast to current tumor diagnostics, a diagnostic which can successfully exploit the acidity of tumors for detection has the potential for detecting very small tumors well before they show overt symptoms, and probably well before they begin to metastasize or cause irreversible organ damage.

3. Distinguish Between Tumor and all Normal Tissues

In order for acidity in the interstitial space of hypoxic areas of tumors to serve as an adequate tumor-specific marker, the detected acidity needs to be essentially unique to tumors. However, there are several normal areas in the body which are at least as acidic as in tumors. Such low-pH areas in the normal body include the lumen of the stomach, often the urine, and muscles undergoing anaerobic metabolism during extreme exercise.

In regard to acidity in the stomach, a diagnostic onco tool which has been seen to be sequestered in acidic areas of tumors in tumor-bearing mice, appears not to label stomach tissue in living mice to any significant extent (unpublished work). This suggests that such compositions, when distributed in the blood and the interstitial space of tissues, do not have significant access to the acidic lumen of the stomach.

In regard to acidic urine (which is in intimate contact with cells lining the proximal tubules of the kidney), there are safe and effective drugs available, such as Acetazolamide, which are known to render the urine basic (pH≧7.4). Thus, the urine of a person can easily and safely be maintained in a basic state for substantial periods of time—more than adequate for the few hours required for a tumor diagnostic procedure using a diagnostic onco tool.

In regard to temporary acidity in muscles during anaerobic exercise, such acidity is easily avoided simply by not engaging in extreme exercise for several hours before and during the diagnostic procedure.

4. Achieve an Adequate Level of Sensitivity

In diagnostics utilizing radioisotopes, when one attempts to detect a very small tumor in a patient it is generally not too difficult to get enough radioisotope into the tumor to generate an adequate signal from that tumor. Instead, typically the greatest challenge is in avoiding excessive signal from the vastly more abundant normal tissues. While modern computer-aided scanning technologies afford greatly increased signal to noise ratios by allowing one to mathematically focus in on each of many small areas within the patient, nonetheless, even with the aid of such technologies it remains that in order to easily detect a small tumor in a reasonable period of time the diagnostic composition should be strongly sequestered in the tumor, and be largely absent from normal tissues.

To satisfy this selectivity requirement, a pH-switch component of the instant invention is designed such that at the pH present in normal tissues (pH 7.4) it exists predominantly in an anionic hydrophilic form which: a) has little or no affinity for tissues; and, b) favors rapid excretion from the body trough the kidneys. Conversely, when the pH-switch enters an acidic area in a tumor the pH-switch undergoes a pH-mediated transition to a non-ionic lipophilic tissue-binding acid form which favors binding to the tumor, with a pS value greater than 6.0 for said pH-switch, where the pS is the pH at the mid-point of the switch from the salt form to the acid form in aqueous physiological saline. Design strategies for, structures of, and methods of use of compositions which provide such selectivity for acidic areas of tumors are described in two pending applications by the inventor, and improvements and extensions in such compositions and methods, developed by the same inventor, are detailed herein.

5. Provide Signal for Detection from Outside the Body

For diagnostic application, one has considerable latitude in selecting the one or more cargo components effective to generate a signal that can be detected from outside the body. Several radioisotopes with favorable properties for use in a cargo component include:

| Radioisotope | Half-life |
|---|---|
| Technetium-99 | 6 hour |
| Iodine-123 | 13 hour |
| Iodine-124 | 4 day |
| Iodine-131 | 8 day |
| Astatine-211 (via Po-211 x-ray) | 7 hour |

B. Therapeutic Application in pH-Switch Strategy

An ideal therapeutic for tumors should: 1) be broadly effective against a wide range of tumor types; 2) be effective against even very small tumors; 3) be highly specific for tumors; and, 4) be highly effective against all cells of the tumors, including both the relatively sensitive fast-dividing cells and the treatment-resistant quiescent cells. While conventional therapies and combinations thereof, including radiation therapy, chemotherapy, and immunotherapy, fail by a large margin to satisfy these requirements, the inventor believes properly designed onco tools, as described hereafter can effectively satisfy this challenging combination of stringent requirements.

1. Treat a Wide Range of Tumor Types

Similar to the case for tumor diagnostics, much of the difficulty in devising a treatment which is effective against a broad range of tumor types has been in identifying some property which is virtually unique to tumors while also being common to a wide range of tumor types, and which can be readily exploited for safe and effective treatment. In contrast to the wide diversity of tumor properties which are currently exploited for treating different tumor types, the inventor believes the acidity in the interstitial space of hypoxic areas of tumors, which appears to be common to most or all tumor types, can be exploited for the treatment of most, and possibly all tumor types.

2. Treat Tumors Having a Wide Range of Sizes

The same considerations which bear on the ability of diagnostic onco tools to detect even very small tumors also apply to the ability of therapeutic onco tools to treat tumors having a wide range of sizes—down to a near-microscopic 1 millimeter in diameter.

3. Avoid Significant Collateral Damage to Normal Tissues

As described in the earlier section on diagnostic application, there are several normal low-pH areas in the body where a therapeutic onco tool might be sequestered, whereupon it could cause damage to surrounding normal tissues. As discussed earlier, it appears that the acidity in the lumen of the stomach is not a problem, and the possibility of low pH in muscles is easily avoided, and low pH of the urine is easily prevented by use of suitable safe and effective over-the-counter drugs.

4. Destroy the Entire Tumor

As noted earlier herein, conventional tumor therapies (radiation and chemotherapy) are reasonably effective in killing the relatively treatment-sensitive fast-dividing cells near tumor capillaries, while those same conventional therapies are generally far less effective against the treatment-resistant quiescent cells in hypoxic/acidic areas more distant from tumor capillaries. Conversely, the onco tools of the present invention are designed to be sequestered only in the acidic areas of tumors populated by treatment-resistant quiescent tumor cells. Thus, if the onco tool is structured to be effective to kill the quiescent cells, then a combination of a conventional tumor therapy to kill the fast-dividing tumor cells, and a therapeutic onco tool to kill the quiescent tumor cells should achieve complete killing of tumors—and probably with substantially less overall toxicity to the patient. This lesser toxicity is a consequence of being able to use a substantially lower dose of radiation or chemotherapeutic because one only needs it to kill the treatment-sensitive population of tumor cells.

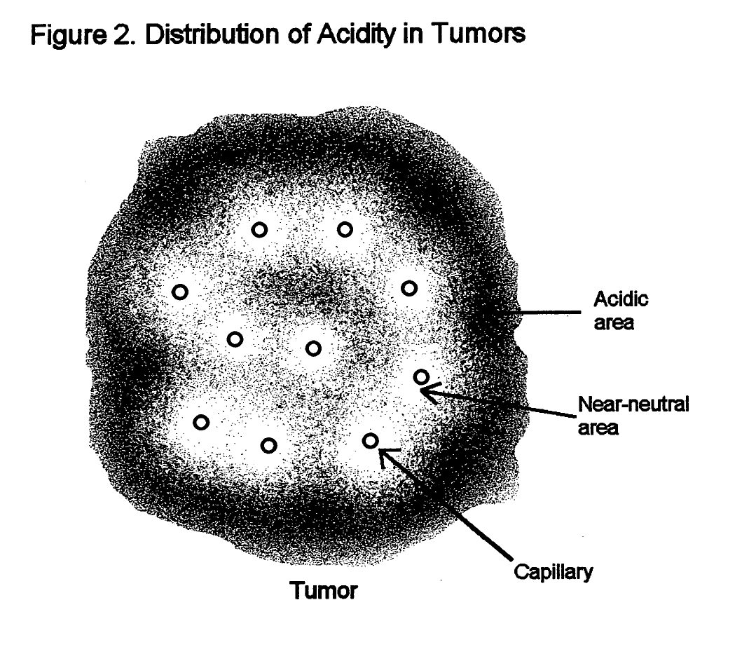

While combining conventional tumor therapies with a therapeutic onco tool offers the prospect of greater safety, higher efficacy, and fewer relapses than conventional therapies alone, the inventor has recently devised an improved therapeutic strategy which entails using only onco tools. The key challenges in this improved onco tool-only strategy are two fold. One challenge is to decisively and completely kill all the treatment-resistant quiescent tumor cells on or in which the onco tool resides. The other challenge is to decisively and completely kill all the treatment-sensitive fast-dividing tumor cells, which may be positioned as far as about 50 to several hundred microns from tissue-associated onco tool, as illustrated in FIG. 2.

The inventor believes these two challenges are best met by using one or more onco tools containing two or more different radioisotopes, where one of the radioisotopes serves to kill the radiation-resistant quiescent tumor cells and one or more other radioisotopes serve to kill the radiation-sensitive fast-dividing tumor cells.

i) The radioisotope used to kill the treatment-resistant quiescent tumor cells should have an emission which releases a vast amount of energy over a very short distance (a few cell diameters), such that the released energy is highly effective to kill all of the radiation-resistant quiescent tumor cells on or in which radioisotope-carrying therapeutic onco tool is positioned. Alpha-emitting radioisotopes appear to best satisfy this demanding requirement. Probably the best radioisotope for this purpose is Astatine 211, which can be generated from natural Bismuth 209 in a medium-energy cyclotron equipped with an alpha particle beam. Astatine has a half-life of 7.2 hours and emits two alpha particles with energies of 5.87 and 7.45 million electron volts, which devastate cells within the approximately 50 to 80 micron paths of the emitted alpha particles (a few cell diameters). Just 1 or 2 such alpha emissions can kill even the most radiation-resistant tumor cell. Actinium-225 is another alpha-emitter with favorable properties for this application.

ii) The radioisotope used to kill the treatment-sensitive fast-dividing tumor cells does not need to have an emission which releases such a high density of energy along its path, but the radioisotope's emission does need to release its energy over a substantially longer path. This is because the emitting radioisotope will be positioned in the acidic area of the tumor while its emitted radiation needs to kill fast-dividing tumor cells close to tumor capillaries, and such fast-dividing cells can be as far as about 50 to several hundred microns from any acidic area of the tumor, and hence as far as about 50 to several hundred microns from any isotope-carrying therapeutic onco tool. A beta-emitting radioisotope, or a combination of two or more different beta-emitting radioisotopes with differing path lengths, should satisfy this requirement.

One has considerable latitude in selecting the beta-emitting radioisotope or combination of beta-emitting radioisotopes which are to serve for killing the fast-dividing tumor cells. A few of the promising radioisotopes for this purpose include:

| | | Approximate | |
|---|---|---|---|
| | | Mean | |
| Radioisotope | Half-life | Path Length | |
| Bromine-82 | 35 hours | | |
| Iodine-131 | 8.1 days | 910 | microns |
| Rhenium-186 | 3.8 days | 1,800 | microns |
| Yttrium-90 | 2.7 days | 3,900 | microns |

It should be noted that because of the complex effects cell killing may have on tumor acidity, it may be desirable to deliver the onco tool containing the alpha-emitting radioisotope either well before or well after the time the onco tool containing the beta-emitting radioisotope, or the set of onco tools containing the set of beta-emitting radioisotopes, is delivered.

### Definition of Terms Used in the Present Invention

The terms used herein have the following specific meanings, unless otherwise noted.

Onco Tool means a composition that includes at least one pH-switch component and at least one cargo component.

Diagnostic Onco Tool means an onco tool which includes a cargo component effective to report the presence of the onco tool.

Therapeutic Onco Tool means an onco tool which includes a cargo component effective to kill cells.

Acidic Area of Tumors: means an area of a tumor where the interstitial space has a pH of less than 7.2.

pH-Switch means a structural component which at the pH present in normal tissues (pH 7.4) exists predominantly in an anionic hydrophilic form which: a) has little or no affinity for tissues; and, b) favors rapid excretion from the body through the kidneys. When that pH-switch component enters an acidic area of a tumor it undergoes in substantial part a pH-mediated switch to a non-ionic lipophilic acid form which is sequestered in the tumor by virtue of binding to extra-cellular structures and/or binding to cell surfaces and/or entering into cells.

pH-Switch Peptide means a peptide having the following properties:
i) a length of about 14 to about 50 amino acids;
ii) about 20% to about 40% of the amino acids are glutamic acids;
iii) the glutamic acid side chains are dispersed around the helical axis and along its length;
iv) at least 90% of the non-acid amino acids are lipophilic amino acids selected from the group consisting of leucine, isoleucine, norleucine; and methionine; and,
v) substantially all of the amino acids are of the same chirality.

Improved pH-Switch Peptide means a peptide having the following properties:
i) a length from about 14 to about 50 amino acids;
ii) about 20% to about 34% of the amino acids are glutamic acids;
iii) the glutamic acid side chains are not congregated on a face or an end of the alpha helix, but instead are dispersed around the helical axis and along the length of the helical axis;
iv) at least 90% of the non-acid amino acids of the pH-switch peptide are lipophilic amino acids selected from the group consisting of: leucine, isoleucine, norleucine, and methionine;
v) at least one terminus of the pH-switch peptide is predominantly anionic at pH 7.4, and in substantial part non-ionic at a pH above 6.0 by virtue of having a carboxylic acid-containing group having a pKa value greater than 4.0 and positioned at or within one amino acid from the end of the pH-switch peptide;
vi) substantially all of the amino acids are of the same chirality;

Advanced pH-Switch means a substance having the following properties:
i) contains an aliphatic ring structure selected from the group consisting of: 4-membered rings, 5-membered rings, and 6-membered rings;
ii) contains a carboxylic acid moiety directly linked to said aliphatic ring structure;
iii) said carboxylic acid moiety is separated from any linked electron-withdrawing group by at least two carbons;
iv) contains an H-bond acceptor moiety selected from the group consisting of:
   a) part of said aliphatic ring structure;
   b) directly linked to said aliphatic ring structure; and
   c) linked through one atom to said aliphatic ring structure;
v) said H-bond acceptor moiety has a structure which cannot serve as an H-bond donor moiety;
vi) said carboxylic acid moiety and said H-bond acceptor moiety are positioned in close proximity and are properly positioned and oriented wherein they can form an internal acid-specific H-bond.

Cargo means a structural component of the onco tool which serves to bind a radioisotope which is effective to report the presence of the onco tool, or which is effective to kill cells. The cargo component can exist in either of two forms. In the first form the cargo component has a structure which is capable of readily binding to a selected radioisotope, but has not yet bound an isotope. An onco tool with this form of the cargo component is suitable for shipping and storage. In the second form a radioisotope is bound to the cargo component. An onco tool with this form of the cargo component is delivered into the patent for the purpose of detecting or killing tumors containing acidic areas.

Physiological Conditions means an aqueous solution with a temperature in the range of 20 deg. C. to 40 deg. C. and having a sodium chloride concentration between 0.13 M and 0.17M.

Substantially All means greater than 90%.

Predominantly means greater than 75%.

In Substantial Part means greater than 4%.

Anionic/Hydrophilic Form means a form which carries a negative charge and has an octanol/water partitioning coefficient of less than 0.3.

Non-ionic/Lipophilic Form means a form which does not carry a charge and has an octanol/water partitioning coefficient of greater than 3

Effective to Bind a Radioisotope refers to a form of a cargo component that has a structure which is capable of readily, efficiently, and stably binding a selected radioisotope, but said form of the cargo component has not yet bound such a radioisotope.

Effective to Report the Presence means a radioisotope whose decay within an animal, including a human, generates an emission, such as a gamma ray or positron, that can be readily detected from outside that animal.

pS means the pH value at the mid-point of the switch between the anionic hydrophilic form of a substance and the non-ionic lipophilic form of that substance, where that switch between the two forms is measured with the substance present at 20 milliMolar in aqueous physiological saline (or a close equivalent containing some buffer). While the pS value somewhat resembles the pKa value for a substance, the pS value differs in that it applies to substances whose acid form is strongly lipophilic and so can drop out of solution during titrations, and it is measured in the presence of physiological saline—which has a substantial impact when the acid form of the substance has limited aqueous solubility.

Inventor means James E. Summerton, Ph.D.

### DETAILED DESCRIPTION OF IMPROVEMENTS CONSTITUTING THE PRESENT INVENTION

A. Improved pH-Switch Peptides

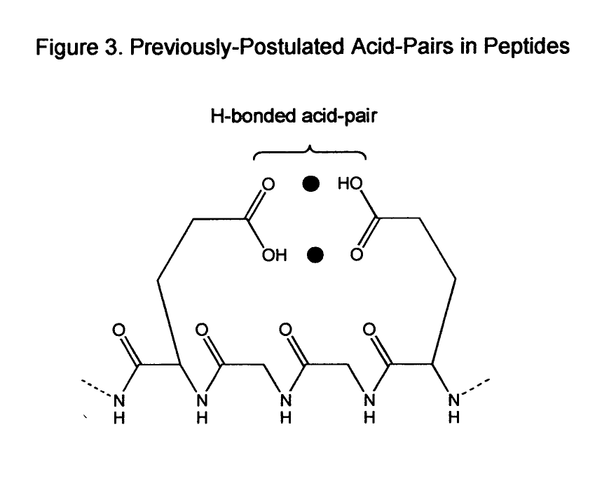

In the 1990s, the inventor developed peptides designed to exist in an anionic/hydrophilic form at neutral pH (pH 7.4), and then convert to a non-ionic/moderately-lipophilic form at the pH within late-stage endosomes (about pH 5.0) (Summerton & Weller, U.S. Pat. No. 6,030,941). In those peptides the glutamic acid plus aspartic acid content ranged from a low of 40% to a high of about 100%. For all such high-acid-content peptides it appeared that results from biophysical studies were compatible with the inventor's postulate that internal H-bonding was driving the acid-mediated transition to the low-pH form. Specifically, the inventor postulated that proximal carboxylic acid side chains were forming double-hydrogen-bonded acid-pairs when such peptides were in the low-pH form, and that it was this internal H-bonding, illustrated in FIG. 3, which was responsible for the unusually sharp pH-mediated transition between hydrophilic and lipophilic forms which occurred at a higher-than-expected pH in aqueous solution (typically between pH 5.0 and 6.0).

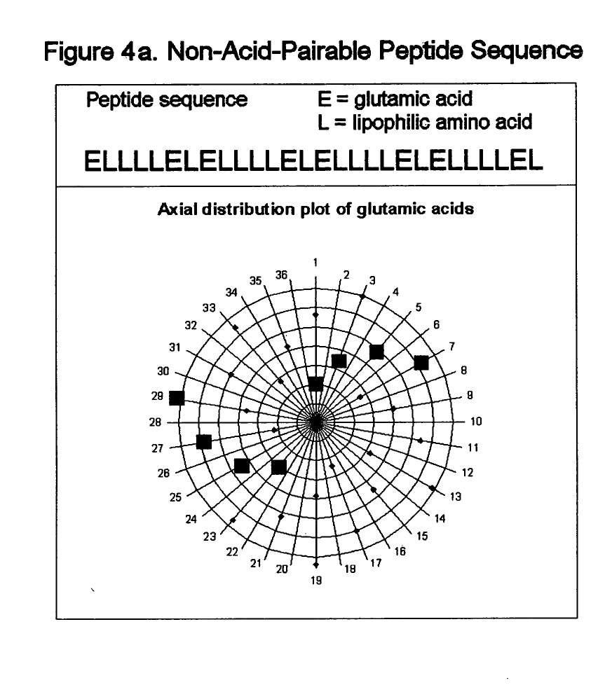

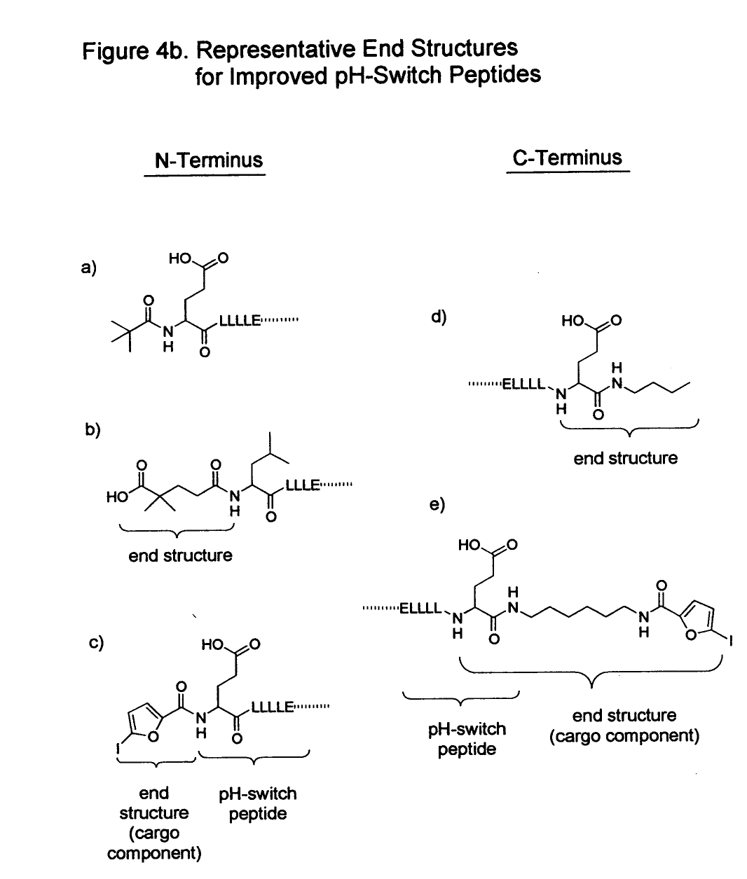

However, more recently when the inventor set out to develop related peptides which would be effective to satisfy the more demanding requirement of converting from their anionic/hydrophilic form at physiological pH (pH 7.4) to a non-ionic/lipophilic form at pH values present in acidic areas of tumors (pH 6.0 to 7.0). In that development effort the inventor found it necessary to restrict the acid side chains to only glutamic acids, and to reduce the glutamic acid content to 40% or less in the core repeating sequence (detailed in a patent application by the present inventor, U.S. patent application Ser. No. 11/069,387, allowed, but not yet published or issued). Still more recently, in the course of continuing investigations on these low-acid-content pH-switch peptides, the inventor prepared a peptide having a sequence that his molecular modeling indicated could not form the postulated double-hydrogen-bonded acid-pairs illustrated in FIG. 3. The amino acid sequence and axial distribution plot of that non-acid-pairable sequence are shown in FIG. 4a.

No Need of Positioning for Acid-Pair Formation

Surprisingly, this peptide, whose acid side chains cannot form the postulated double-hydrogen-bonded pairs, was found to exhibit a transition which is as sharp and occurs at about as high of a pH as similar-composition sequences where the glutamic acid side chains were properly positioned to form the postulated double-hydrogen-bonded acid-pairs. This finding demonstrates that positioning of the acid side chains for hydrogen-bonded pairing of their carboxyl moieties is not necessary in order for a pH-switch peptide to undergo a relatively sharp pH-mediated transition at a pH greater than 6.0. In fact, experimental results have been published which could lead one to suspect that those previously postulated double-H-bonded acid pairs probably never form in aqueous solution (Rebec et al., J. Amer. Chem. Soc. vol 108 pages 6068-6069 (1986)).

While the inventor now recognizes that the previously-postulated acid-pair formation is not necessary, and probably never occurs, in pH-switch peptides, nonetheless, the previously determined requirement remains that the glutamic acid side chains not be congregated on one face or at one end of the peptide's alpha helix, but instead be well dispersed around the helical axis and along the helical axis. This is essential in order to avoid lipophilic patches of a size sufficient to cause aggregation of the peptide, or sufficient to cause tissue binding by the peptide when that peptide is in its anionic form at pH 7.4. It is noteworthy that absent the earlier constraint of having to position glutamic acid side chains for acid-pair formation, one gains significant latitude for selecting sequences wherein those glutamic acid side chains have an improved distribution around and along the helical axis of the peptide.

Avoid Terminus which Remains Lipophilic at pH 7.4

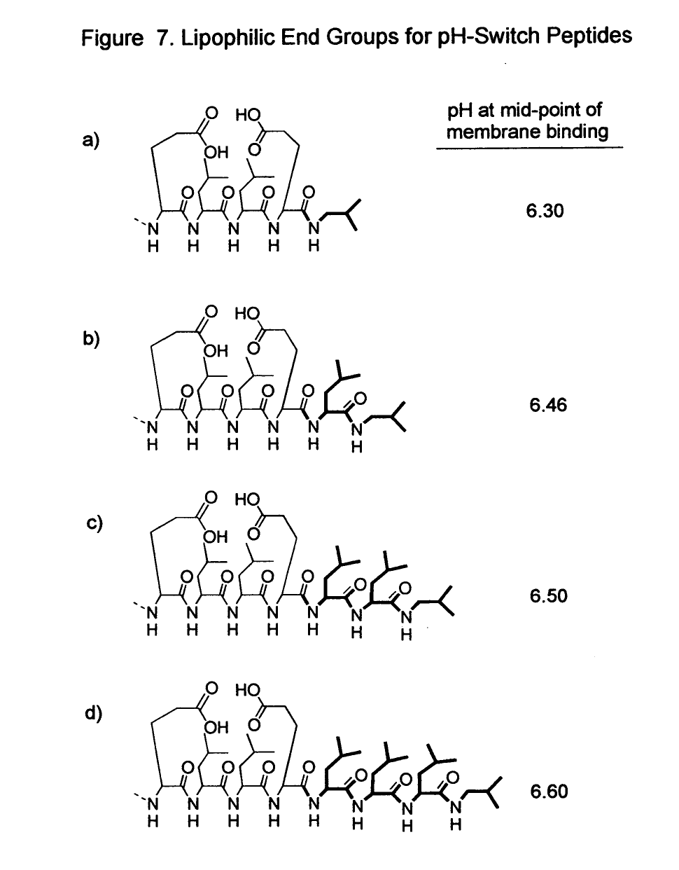

Conventional peptides having core repeating amino acid sequences suitable for use as a pH-switch exhibit poor binding to tissues when such peptides are in their low-pH form. This is because in the critical pH range between 6.0 and 7.0 their ends remain ionic and hydrophilic. To remedy this, in earlier pH-switch peptides the inventor has added a structure to at least one end of the peptide which renders that end non-ionic and lipophilic. Such added end structures, which have been referred to as entry ends in the pending patent applications (patent application Ser. Nos. 11/069,849, pending; and 11/069,387, allowed but not yet published or issued), have been shown to be effective in raising the pH at which a pH-switch peptide binds to isolated cell membranes. FIG. 7 shows several representative end structures, all with the same core peptide sequence, along with the respective experimentally-determined mid-point pH values for their binding to isolated cell membranes. The progressive increase in the mid-point pH values seen with increasing lipophilicity of the end structure demonstrates that by using a relatively lipophilic end structure one can achieve a desirable increase in the pH at which the peptide binds to cell membranes.

While these added end structures were found to give a desirable increase in the pH at which the pH-switch peptides bind to isolated cell membranes, nonetheless, in more recent studies in tumor-bearing mice it was found that adding an excess of lipophilic groups at the end of the peptide, particularly as in structure (d) of FIG. 7, caused the peptide to bind to both tumors and normal tissues. This suggests that this lipophilic end structure is dominating the association of the pH-switch with tissues, and because that end structure is equally lipophilic at both the acidic pH in tumors and the neutral pH of normal tissues, peptides with such lipophilic end groups lack the crucial property of not binding to tissues at the pH of normal tissues.

Thus, in earlier work it was found that increasingly lipophilic end modifications appeared to be desirable in order to give a higher and more practical level of tissue binding in acidic areas of tumors However, more recent studies have shown that end structures which remain non-ionic and lipophilic at pH 7.4 also cause unacceptable binding to normal tissues in tumor-bearing mice.

New End Structures Giving Improved pH Discrimination

In view of the problem presented by the earlier end structures that remain non-ionic and lipophilic at pH 7.4, the inventor has devised new end structures which reduce the specificity problem associated with earlier end structures. Such new end structures entail:

a) capping the peptide end with a moderately lipophilic moiety, such as a pivalate on the N-terminus, or a butyl amine on the C-terminus;

b) positioning a carboxylic acid-containing group, having a pKa value greater than 4.0, at or within one amino acid from the peptide end;

c) selecting the peptide sequence such that at least one, but preferably four, of the amino acids next to that carboxylic acid-containing terminal group are lipophilic amino acids selected from leucine, isoleucine, norleucine, and methionine.

It is the incorporation of a terminal carboxyl group that is ionic and hydrophilic at pH 7.4 which helps to avoid the loss of specificity seen with pH-switch peptides containing the previously-used end structures. FIG. 4b illustrates representative peptide end structures that satisfy these new design criteria.

In light of the inventor's recent experimental finding that peptides with non-pairable carboxyls can serve as effective pH-switches, the inventor prepared and tested a variety of new peptide sequences, now without the sequence constraints imposed by the previously believed need to position glutamic acid side chains for acid-pair formation. From the test results with those new peptides he has revised the design criteria for pH-switch peptides, as follows:
i) a length from about 14 to about 50 amino acids;
ii) about 20% to about 34% of the amino acids are glutamic acids;
iii) the glutamic acid side chains are not congregated on a face or an end of the alpha helix, but instead are dispersed around the helical axis and along the length of the helical axis;
iv) at least 90% of the non-acid amino acids of the pH-switch peptide are lipophilic amino acids selected from the group consisting of: leucine, isoleucine, norleucine, and methionine;
v) at least one terminus of the pH-switch peptide is predominantly anionic at pH 7.4, and in substantial part non-ionic at a pH above 6.0 by virtue of having a carboxylic acid-containing group having a pKa value greater than 4.0 and positioned at or within one amino acid from the end of the pH-switch peptide;
vi) substantially all of the amino acids are of the same chirality;

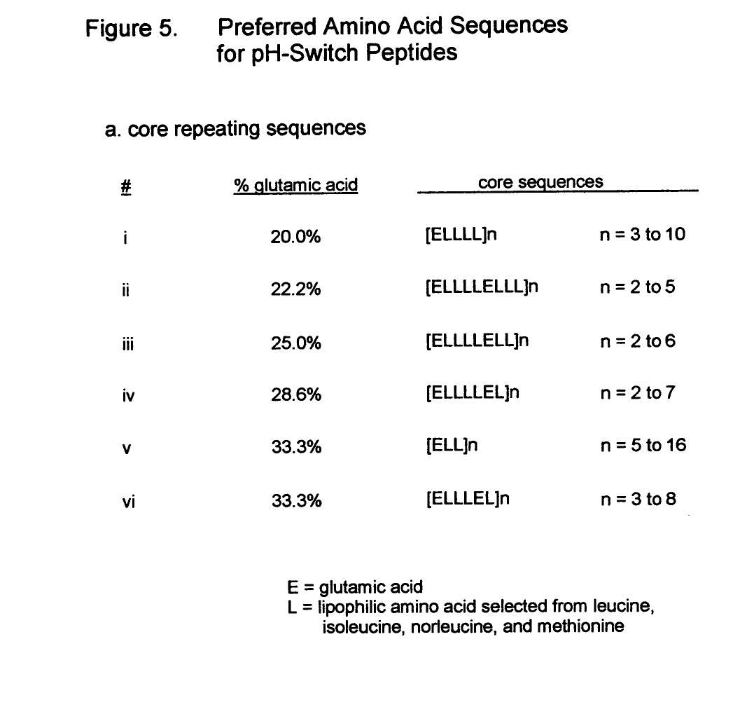

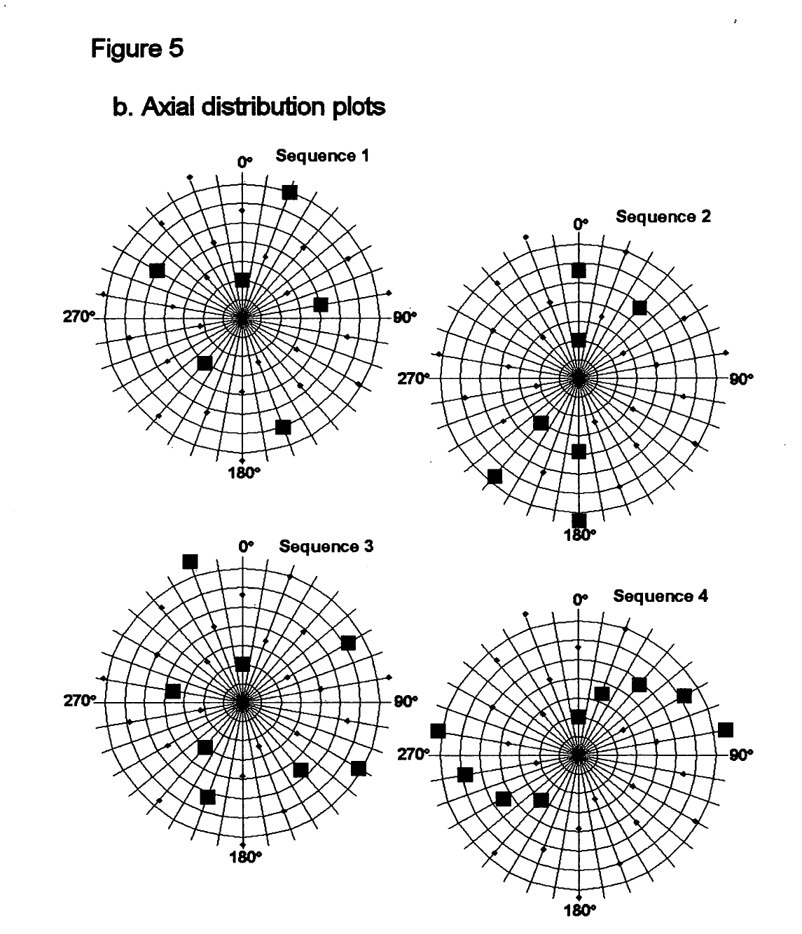

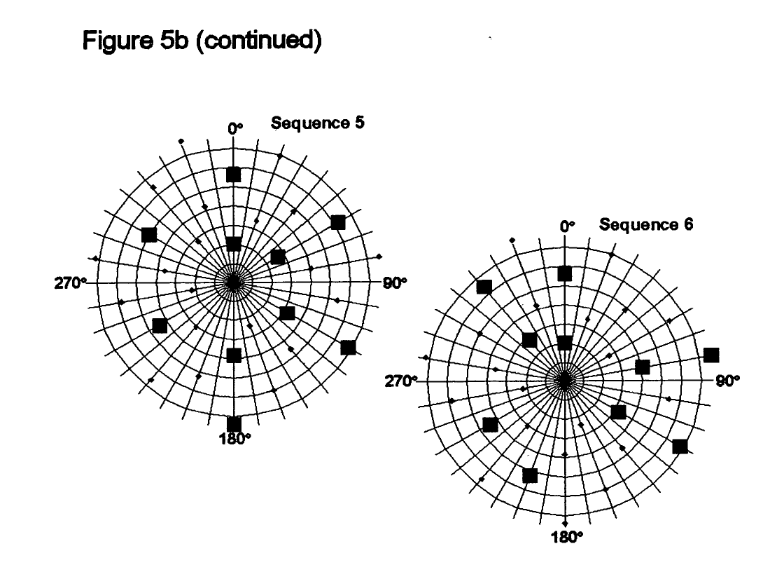

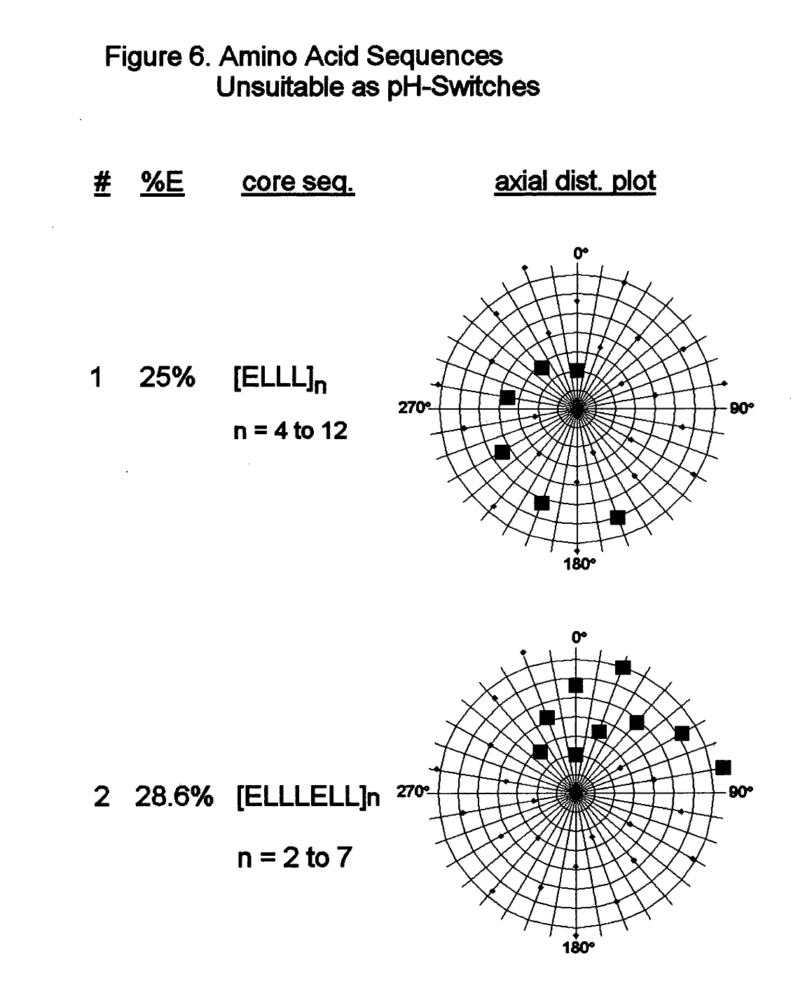

Based on these updated design criteria for pH-switch peptides, six preferred core repeating amino acid sequences have been identified. These six core repeating sequences are noteworthy in that, compared to earlier pH-switch peptides, the new sequences are generally lower in glutamic acid content, and this lower glutamic acid content serves to compensate for the lesser lipophilicity of the newly designed end structures, compared to earlier end structures. These six core repeating sequences also generally afford an improved dispersion of the glutamic acid side chains around and along the peptide's helical axis, which is a consequence of no longer limiting sequences to those which were predicted to form acid-pairs. FIG. 5 shows these six preferred repeating amino acid sequences and their respective axial distribution plots. These sequences are suitable for pH-switch peptides because they show good aqueous solubility at pH 7.4 and little or no binding to biological structures, but when having a suitable end structure they can undergo the desired transition to a lipophilic tissue-binding form at a pH value above 6.0. Note that repeating peptide sequences i and iv in this figure have acid side chain distributions which are incompatible with forming any of the previously-postulated doubly-hydrogen-bonded acid pairs, and most of the core repeating peptide sequences in this figure are incompatible with forming at least some of the postulated doubly-hydrogen-bonded acid pairs previously believed to be essential for effective pH-switch activity. For comparison, FIG. 6 shows several core repeating amino acid sequences which are not suitable for pH-switch peptides because the glutamic acid side chains are congregated largely on one face of the alpha helix, resulting in inadequate aqueous solubility of the peptides at pH 7.4.

It should be noted that peptides containing these six preferred repeating amino acid sequences in FIG. 5, and having ends as specified in the above revised design criteria for pH-switch peptides, differ from previous sequences claimed in applicant's pending patent application (patent application Ser. Nos. 11/069,849, pending; and 11/069,387, allowed but not yet published or issued). This divergence from the prior claimed sequences arises because the new sequences in FIG. 5 of the present invention were not designed to adhere to the restrictions in the previous patent applications relating to pair formation by acid side chains, and so the new sequences commonly fail to satisfy those earlier limitations on allowable sequences. Nonetheless, peptides with these new sequences shown in FIG. 5, referred to as “improved pH-switch peptides”, have been found to exhibit excellent properties with respect to their pH-mediated transition between salt and acid forms.

B. Advanced pH-Switches

In an effort to further improve discrimination between acidic areas of tumors and normal tissues, the inventor recently began efforts to devise advanced pH-switch structures which he hoped would undergo the desired pH-dependant shift between salt and acid forms, and provide a particularly large shift in the hydrophilicity/lipophilicity properties between the salt and acid forms. The inventor hoped that such advanced pH-switches affording an enhanced shift in hydrophilicity/lipophilicity properties could be used to further increase the specificity for acidic areas of tumors. It was also envisioned that such advanced pH-switch structures, combined with a suitable cargo component, might serve on their own as onco tools for detection and treatment of tumors.

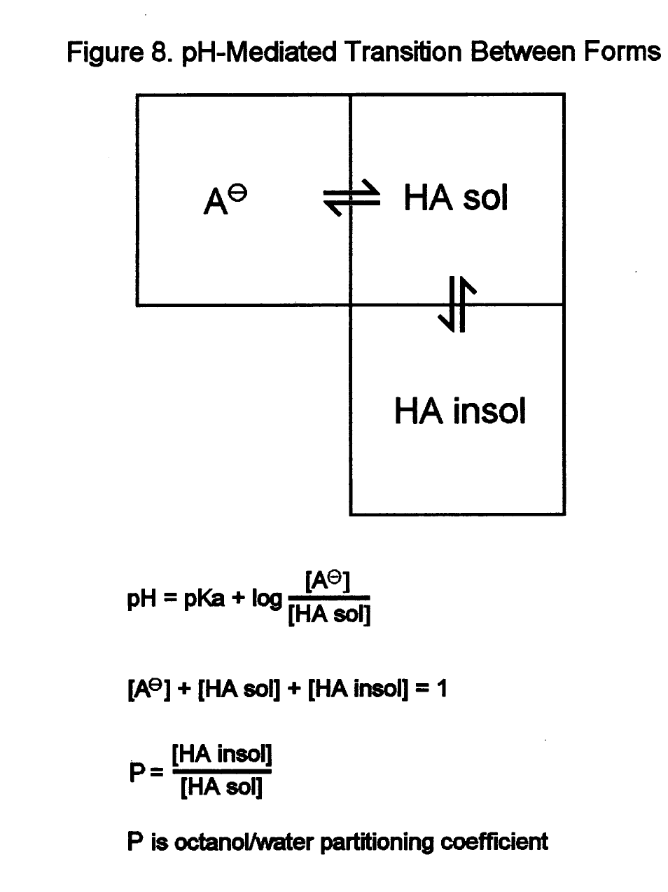

The key challenge remained to devise an end structure for pH-switch peptides, or a stand-alone pH-switch structure, which at pH 7.4 has good aqueous solubility and is anionic, but then switches in substantial part to a form which is non-ionic and lipophilic at a pH present in acidic areas of tumors. To obtain a quantitative measure of how lipophilic the acid form should be, mathematical modeling was carried out to calculate the pH-dependent transition between forms for a weak-acid substance which exists at high pH in an anionic water-soluble form, [A−], and which converts at a lower pH to a non-ionic lipophilic form which has some aqueous solubility, [HA sol], and which can also become water-insoluble by virtue of precipitating or oiling out, or binding to tissue components, or partitioning into a lipophilic phase such as octanol or a cell membrane. This insoluble fraction is denoted [HA insol]. The inter-conversions of these three forms, along with the key equations used in the mathematical modeling, are shown in FIG. 8. Example 1 details titration experiments which illustrate this mathematically-predicted impact of the lipophilicity of the acid form on the pH-dependent transition from the salt form to the acid form.

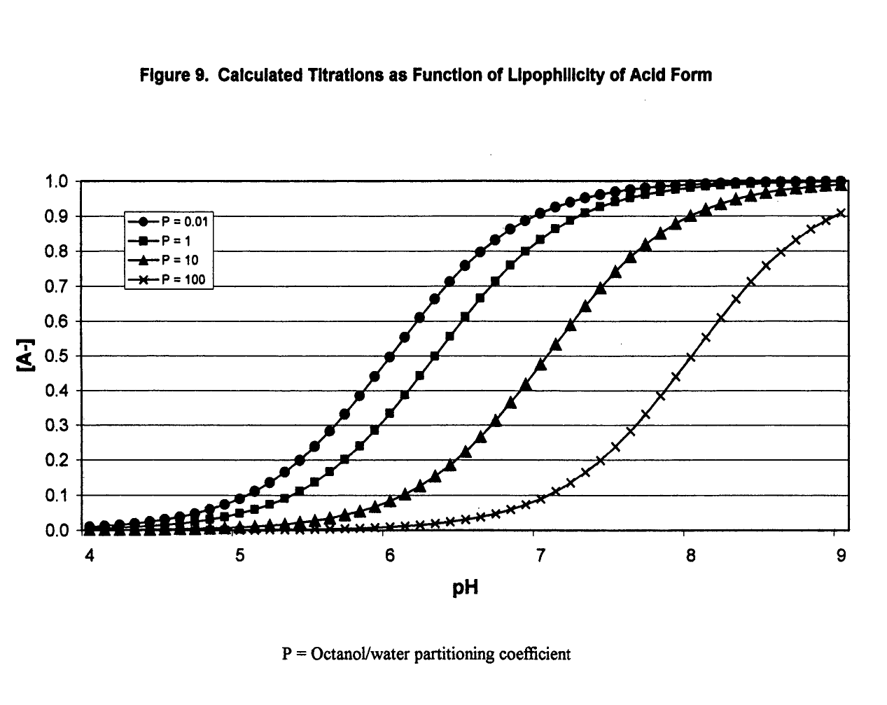

The results from that modeling, shown in FIG. 9, suggest that the pH of the transition between the salt and acid forms is effectively raised by 1 pH unit for each 10-fold increase in the octanol/water partitioning coefficient of the non-ionic lipophilic acid form. However, such an increase will only apply after the acid form reaches the lipophilicity threshold effective to initiate precipitation or tissue binding or partitioning into a non-polar phase.

In light of the impact increased lipophilicity of the acid form can have on the pH of the transition between the salt and acid forms of a substance, it appeared that it might be possible to devise a structure which would show an increased ability to discriminate between tumors and normal issues by designing it in such a manner that the structure's salt form is more hydrophilic and/or its acid form is more lipophilic than is the case for conventional carboylic acids. Accordingly, the inventor recently began a quest to devise structures explicitly designed to undergo a greater pH-dependant shift in their hydrophilicity/lipophilicity ratio than is the case for conventional carboxylic acids.

1. Internal Acid-Specific H-Bond

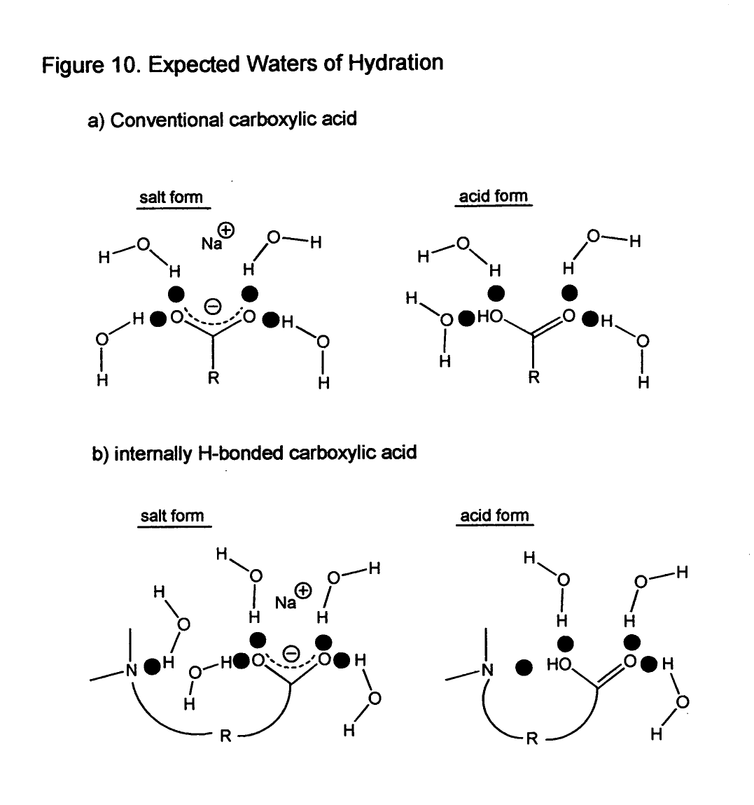

With the objective of improved discrimination between tumor and normal tissues, the inventor postulated that an exceptionally large shift in the hydrophilicity/lipophilicity ratio between the salt and acid forms might be achieved with a structure which can form an internal hydrogen bond. In regard to this predicted enhanced shift in the hydrophilicity/lipophilicity ratio between the salt and acid forms for such an internally-H-bonding structure, FIG. 10a illustrates the expected waters of hydration directly H-bonded to a conventional carboxyl in its salt form and in its acid form, and FIG. 10b illustrates corresponding expected waters of hydration directly H-bonded to the salt and acid forms of a postulated structure effective to form the desired internal H-bond.

Clearly, both the conventional carboxyl and the carboxyl effective to form an internal H-bond lose their counter-ion in going from the salt form to the acid form. However, while the conventional carboxyl is expected to have the same number of waters directly H-bonded in both the salt form and the acid form, the inventor believes that for a carboxyl able to form an internal H-bond there will be a net loss of several (probably two) directly-H-bonded waters when going from the salt form to the acid form. The inventor postulated that this loss of about two waters of hydration should afford an enhanced shift in the hydrophilicity/lipophilicity ratios for the salt and acid forms, relative to this ratio for a similar structure which does not form such an internal H-bond. The inventor also postulated that such an internal hydrogen bond should favor the transition to the acid form, resulting in an increase in the pKa value for the carboxylic acid moiety of the structure. These factors were predicted to cause a significant increase in the pS value for advanced pH-switches capable of forming an internal H-bond in aqueous solution.

Conventional wisdom among experts in hydrogen bonding typically holds that a lone hydrogen bond will not be stable in an aqueous environment because of competition with water's vast concentration of H-bond acceptor sites (110 Molar) and H-bond donor sites (110 Molar). Instead, it is generally believed that a stable non-covalent interaction in aqueous solution requires a multiplicity of interactions, selected from H-bonds, hydrophobic interactions, and electrostatic interactions.

While this requirement for a multiplicity of non-covalent bonds may well be true for intermolecular interactions, the inventor suspected that, contrary to the conventional wisdom, it might be possible to devise compact structures which will form a single relatively stable pH-dependent intramolecular H-bond in aqueous solution, where that H-bond both serves to favor the acid form over the salt form, and is effective to increase the lipophilicity of the acid form. The inventor predicted that the combination of these two factors should result in a significant increase in the pS value. The crucial question then was: could practical structures be devised which would form such an internal H-bond in aqueous solution?

After considerable experimentation, novel structures have been devised which appear to form the desired single pH-dependent intramolecular H-bond. As predicted, this internal H-bond appears to significantly enhance the shift in the hydrophilicity/lipophilicity properties in going from the salt form to the acid form, as well as raising the pH at which the structure switches from its anionic/hydrophilic non-tissue-binding form to its non-ionic/lipophilic tissue-binding form, relative to corresponding properties which would be expected for a conventional carboxylic acid. The inventor uses the term “advanced pH-switch” to mean a pH-switch which is designed to form an internal H-bond in aqueous solution, where that internal H-bond enhances the shift in the hydrophilicity/lipophilicity ratio between the salt and acid forms of the structure.

Results from molecular modeling and from experimental work detailed in the examples section later herein suggest that the following three properties are essential in order for an advanced pH-switch to form a suitable and adequate internal H-bond in aqueous solution.

a) Acid-Specific H-Bond

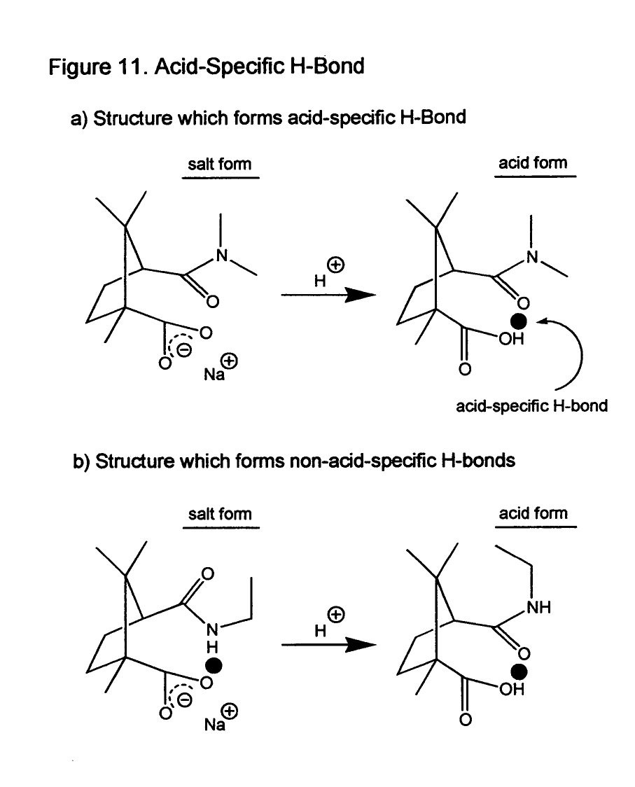

The structure must contain a carboxyl moiety which is positioned in suitable proximity to an H-bond acceptor moiety for formation of an H-bond. When that carboxyl moiety is in its acid form it must serve as the H-bond acceptor, and the proximal H-bond acceptor moiety must be such that it can only serve as an H-bond acceptor, and cannot serve as an H-bond donor. The inventor refers to an H-bond formed by such a structure as an “internal acid-specific H-bond.” FIG. 11a shows a structure which can form only an internal acid-specific H-bond. Conversely, FIG. 11b illustrates a similar, but unacceptable structure which can form both an internal H-bond when the carboxyl is in its acid form and an internal H-bond when the carboxyl is in its salt form. The inventor calls this dual H-bonding capability “internal non-acid-specific H-bonding,” and his experimental results indicate that such non-acid-specific H-bonding is unacceptable because it fails to provide an adequate increase in the pS value, and it fails to afford the desired increase in the lipophilicity of the acid form. Example 2 describes experimental evidence that in acidic conditions the structure in FIG. 11a forms an internal acid-specific H-bond in aqueous solution, as well as evidence that said internal acid-specific H-bond causes a desired increase in the pH of the transition between salt and acid forms. Conversely, this same example also shows that the very similar structure in FIG. 11b, designed to form non-acid-specific H-bonds, fails to provide that desired increase in the pS value, nor does its acid form show the dramatic aqueous insolubility (i.e., lipophilicity) seen with the acid-specific internal H-bonding structure in FIG. 11a.

B) Minimal Conformation Freedom Between H-Bonding Moieties

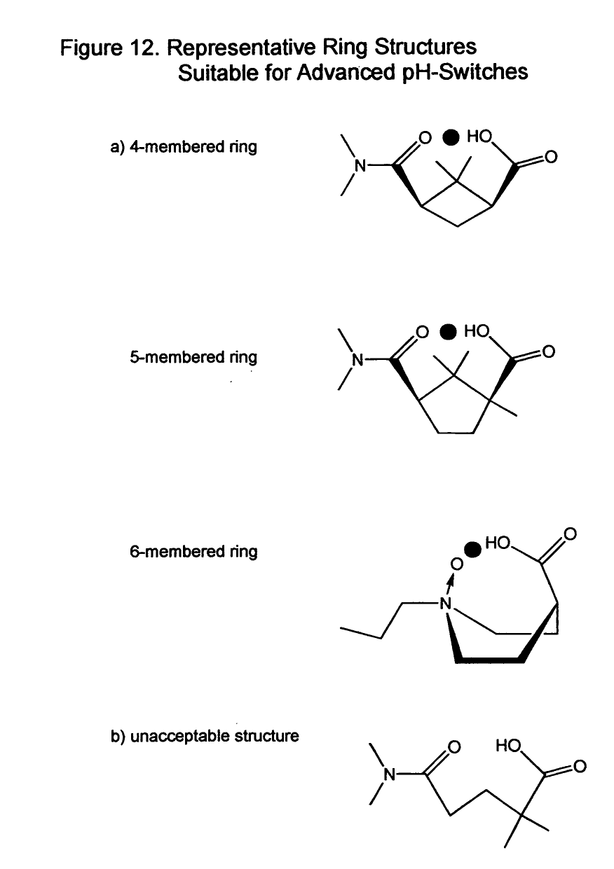

The H-bond acceptor moiety and the carboxyl serving as the H-bond donor moiety should be held in close proximity by a structure which has minimal conformational freedom. This limited conformational freedom can be achieved by using a suitable ring structure. Molecular modeling and early experimental work suggests that 4-membered, 5-membered, and 6-membered aliphatic rings are preferred for this purpose. FIG. 12a shows representative 4-membered, 5-membered and 6-membered aliphatic ring structures which allow only very limited conformational freedom between the H-bond donor (OH of the carboxylic acid) and suitably-positioned H-bond acceptor moieties. Conversely, FIG. 12b shows a somewhat similar structure which is unacceptable because its acyclic structure allows excessive conformational freedom between the H-bond donor and H-bond acceptor moieties. Example 3 describes experimental evidence showing that a structure with minimal conformational freedom between H-bonding moieties can afford the desired increase in the pS value, while a similar structure with substantially greater rotational freedom between H-bonding moieties fails to provide that desired increase in the pS value—and so is presumably failing to form an adequately stable internal acid-specific H-bond.

C) Carboxyl Insulated from Inductive Effects of Electron-Withdrawing Groups

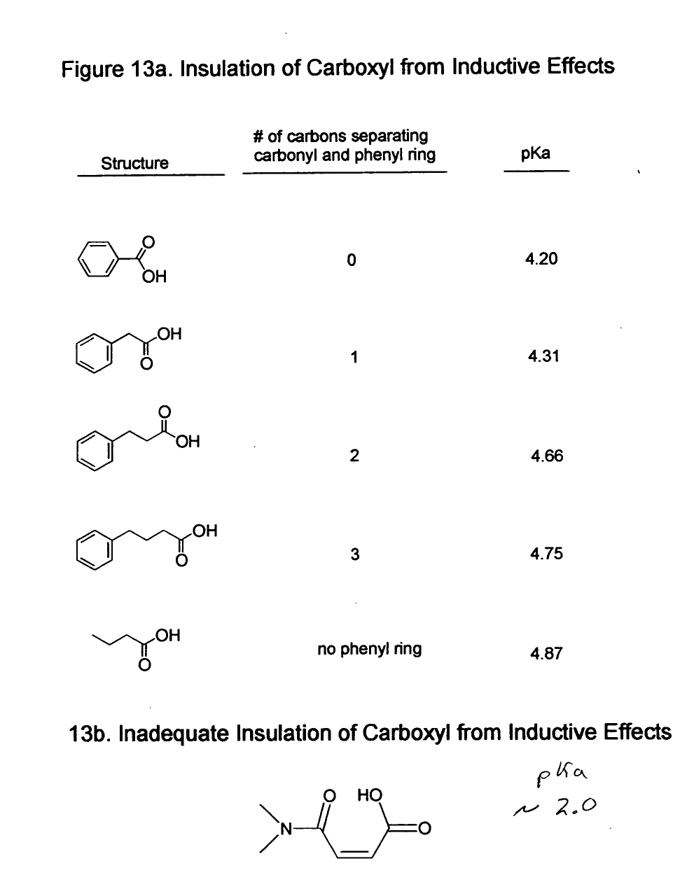

The carboxylic acid which is to serve as the H-bond donor moiety should be separated from any linked electron-withdrawing group by at least two, and preferably three or more carbons. This avoids any substantial reduction in the pKa value of that carboxyl due to inductive effects from electron-withdrawing groups. The molecular structures and their corresponding pKa values shown in FIG. 13a illustrate how an increasing number of aliphatic carbons can progressively insulate a carboxyl from the pKa-reducing effects of a phenyl group. FIG. 13b shows a structure wherein inductive effects from the proximal amide moiety causes an undue reduction in the pKa of the carboxylic acid moiety. Example 4 describes experimental evidence indicating that even with minimal conformational freedom between H-bond donor and acceptor moieties, if there is inadequate insulation of the carboxyl from inductive effects the pH of the transition between salt and acid forms will be too low to be useful in a pH-switch.

In addition to the above 3 essential properties, it appears that at least one additional property, selected from the following two properties, is desirable to assure formation of an adequate internal H-bond in aqueous solution.

D) Lipophilic Groups Partially Shielding H-Bonding Site

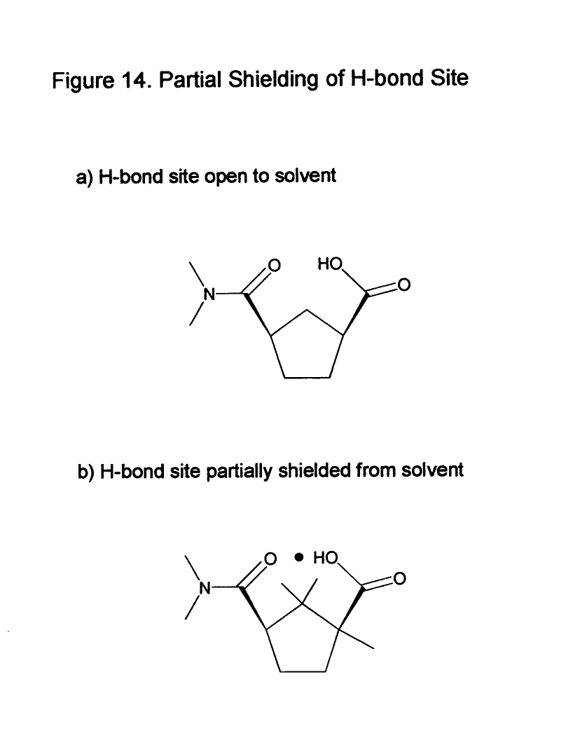

A principal challenge in forming a lone H-bond in an aqueous environment is to preferentially form that H-bond in the presence of the vast concentration of competing H-bond donors and H-bond acceptors of the surrounding water. Based both on biochemical studies of enzyme catalytic sites and on the inventor's molecular modeling, he postulated that the desired intramolecular H-bond might be more favored if the H-bonding site were partially shielded from the bulk water by the presence of lipophilic groups. FIG. 14 shows two structures, one where the H-bonding site is more open to the solvent, and the other where that H-bonding site is partially shielded from the solvent by an adjacent methyl group. Subsequent syntheses and titration studies, detailed in Example 5, provide evidence for the value of such partial shielding from the solvent as a means for favoring the desired intramolecular H-bond.

e) Low-Barrier H-Bond

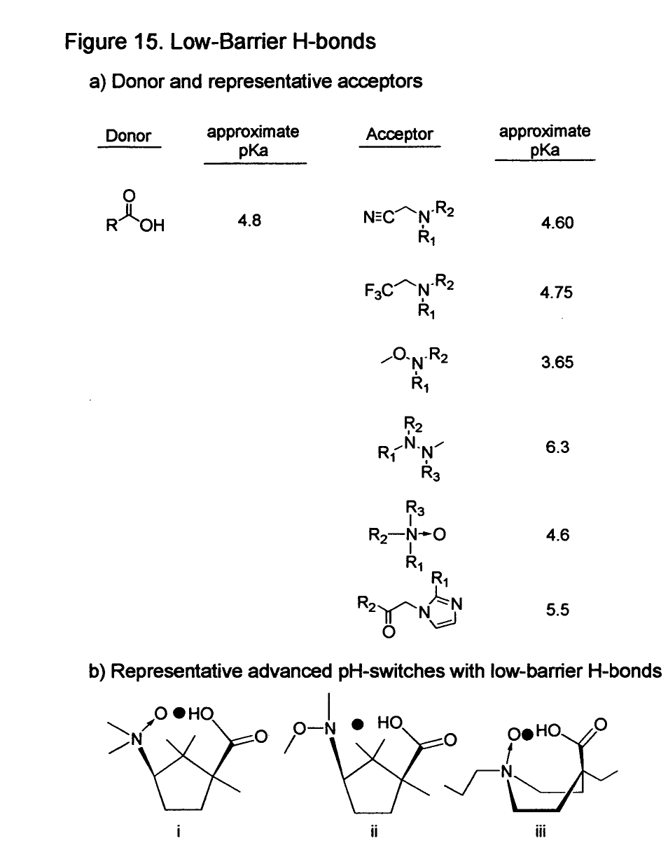

The term “low-barrier H-bond” is used herein to mean a non-covalent bond formed between an H-bond donor moiety and an H-bond acceptor moiety, where the pKa values of the two isolated moieties are within about 2 pH units of each other. It should be noted that this definition includes what may also be construed as an internal salt where the hydrogen is closer to the acceptor moiety than to the donor moiety. Such low-barrier H-bonds are generally found to be exceptionally strong and so can appreciably favor the desired intramolecular H-bond in pH-switches. FIG. 15 shows several H-bonding moieties which are appropriate for forming low-barrier H-bonds in advanced pH-switches, and also illustrates several representative pH-switch structures containing appropriate H-bonds of the low-barrier type. Example 6 describes the synthesis and testing of a representative pH-switch (the N-oxide of N-propylisonipocotic acid), along with experimental evidence for formation of the desired low-barrier H-bond in aqueous solution.

2. Structural Optimization

As noted previously, the crucial requirements for both a pH-switch and an advanced pH-switch are that the structure's anionic salt form be sufficiently hydrophilic that it has little or no affinity for tissues, while its non-ionic acid form be sufficiently lipophilic that it is effectively sequestered in tissues. While the internal acid-specific H-bond of an advanced pH-switch, detailed above, makes a substantial contribution to meeting these two requirements, it is also generally necessary to further optimize the structure of a prospective advanced pH-switch to best meet these requirements.

The essential challenge in optimizing an advanced pH-switch is to incorporate into the structure sufficient lipophilic groups (generally alkyl groups) to give an adequately high pS value, without introducing so many lipophilic groups that one gets undue tissue binding at pH 7.4. While molecular modeling on a computer with commercially-available modeling programs can provide some guidance toward such optimization, efforts along this line to date suggest that most computer-generated predictions relating to pH-dependent properties in aqueous solutions are of limited accuracy. It is particularly difficult to model the solubility properties of a weak-acid substance whose acid form is quite lipophilic, and wherein there is a substantial concentration of salt in the aqueous solution. Thus, it appears the best approach is to design and synthesize a series of closely related structures and then empirically assess their properties in appropriate test systems.

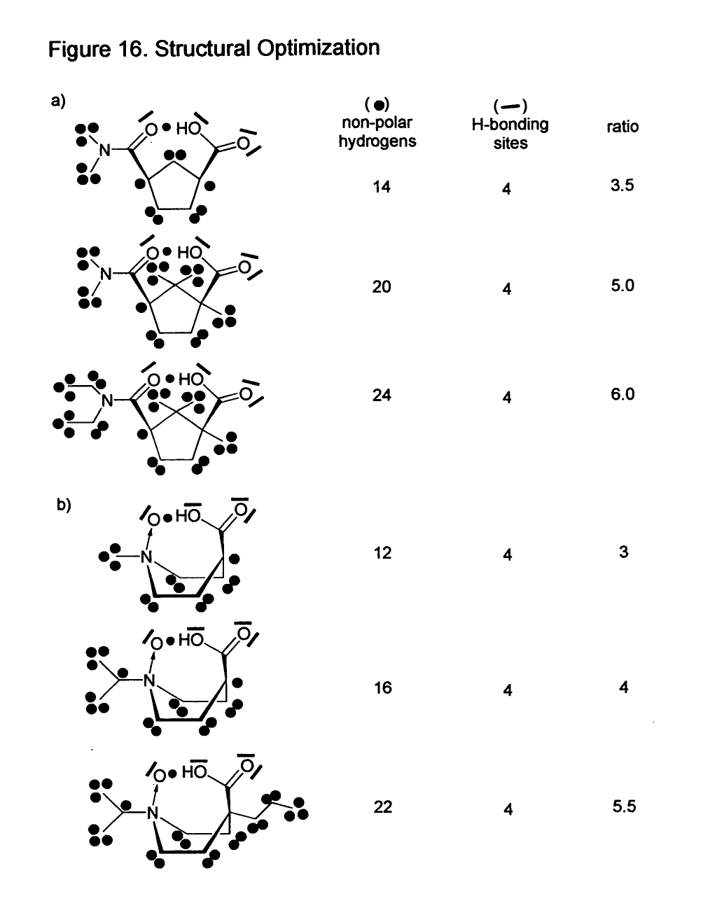

One simple design approach that has provided useful guidance in such optimizations entails first drawing a prospective advanced pH-switch structure designed to form an internal acid-specific H-bond. Next, one counts the number of non-polar hydrogens exposed to the solvent, and counts the number of expected H-bonding sites in the acid form of that structure. The inventor's past experience suggests that the ratio or non-polar hydrogens to H-bonding sites should range from a minimum of about 3.0 to a maximum of about 7.0, and preferably be in the range of about 4.0 to about 6.0. Generally, it is desirable to design a series of related structures with various non-polar alkyl groups added so as to give multiple structures having ratios of non-polar hydrogens to H-bonding sites within the preferred range. It is also desirable that the added alkyl groups be selected and positioned so that subsequent syntheses will not require excessive effort. This approach is illustrated in FIG. 16 for two core pH-switch structures.

In regard to initial testing, a simple pH titration of the prospective pH-switch structure in physiological saline is fast and easy and provides useful information about the key properties one is attempting to achieve. A recommended initial test method is detailed in Example 2a. It is also desirable to carry out a study of the pH-dependent partitioning between n-octanol and aqueous buffers ranging from pH 5 to pH 8, as described in Example 2b.

As noted previously, the internal acid-specific H-bond in an advanced pH-switch structure is predicted to contribute to an increase in the pS value by two mechanisms: 1) formation of the acid-specific H-bond which directly favors formation of the acid form; and, 2) loss of two waters of hydration during formation of said H-bond causing a substantial increase in the lipophilicity of the H-bonded acid form, which then precipitates or oils out or partitions into a lipophilic environment such as cell membranes. It is of interest to be able to estimate the relative contributions of each of these two mechanisms for a given advanced pH-switch structure. In this regard, it has been found that when one carefully titrates the salt form of a substance whose acid form precipitates or oils out of aqueous solution then one obtains a skewed titration curve with an apparent pKa value appreciably higher than would be expected for such a structure, relative to the titration curve for a similar substance where both the salt and acid forms are fully water soluble. This lipophilicity/insolubility effect is best seen when one plots the first derivative of the titration curve, wherein one commonly sees substantial asymmetry and a shift to higher pH values in the titration plot for the case where the acid form is aqueous insoluble, while one sees a fully symmetrical titration plot at normal pH values for the case where both salt and acid forms are fully water soluble, such as for propionic acid. This lipophilicity/insolubility effect on the titration plot is seen both for substances which cannot form an internal acid-specific H-bond, such as octanoic acid, and for substances which can form an internal acid-specific H-bond, such as the advanced pH-switch structure shown in FIG. 11a.

Luckily, this lipophilicity/solubility effect can be avoided by titrating in a mixed solvent where both the salt and acid forms are fully soluble. A 1:1 mix of methanol and water generally serves for this purpose. In this solvent the first derivative titration curves again become symmetric. In the case of a substance which cannot form a stable internal H-bond, such as octanoic acid, the titration plot now shows a classical pKa value very similar to that seen for a fully water soluble acid, such as propionic acid. However, in the case of a substance believed to form a stable internal acid-specific H-bond, such as the advanced pH-switch structure in FIG. 11a, while the titration plot does become symmetric in methanol/water, nonetheless, the titration plot of such an internal acid-specific H-bonding substance still shows a substantially increased pKa value relative to a very similar structure which cannot form an internal acid-specific H-bond, such as the structure in FIG. 11b.

The above suggests that in titrations in physiological saline the total increase in the apparent pKa value, accompanied by asymmetry in the titration curve, is due to a combination of the H-bond effect plus the lipophilicity/solubility effect described earlier and detailed in FIGS. 8 and 9. Conversely, in titrations in methanol/water a substantial rise in the pKa value for a substance designed to form an internal acid-specific H-bond, relative to the pKa for a very similar substance which cannot form such an H-bond, is principally attributable just to the internal H-bond, and not to any lipophilicity increase in the acid moiety which might be a consequence of that internal H-bond.

In summary, an advanced pH-switch component should have a structure which is effective to form an internal acid-specific H-bond in aqueous solution, where that H-bond serves to raise the effective pKa of the carboxyl moiety and give a greater change in the hydrophilicity/lipophilicity ratio in going from the salt form to the acid form, compared to such properties in a similar substance which cannot form such an internal acid-specific H-bond.

C. Compositions for Improved Tumor Specificity

The interstitial space in normal tissues is generally at or very near pH 7.4, and in acidic areas of tumors a pH of around 6.4 is fairly typical or readily achievable by known methods. If one knows the pKa value of an acid substance (pKa is the pH at which the acid moiety is half ionized in aqueous solution) one can use the Henderson-Hasselbalch equation (pH=pKa+log [A−]/[HA]) to calculate what fraction of that substance will be in the acid form at any selected pH. Table 1 shows the calculated percent of acid substances which will be in the acid form at pH 6.4 (present or achievable in acidic areas of tumors) and at pH 7.4 (in normal tissues), as well as the ratio of the percent in acid form at pH 6.4 divided by the percent in acid form at pH 7.4. These values are given for a representative aliphatic carboxylic acid (propionic acid, pKa 4.87), and for an acid with a moderately high pKa (chlorambucil, pKa 5.80), as well as for two hypothetical advanced pH-switches with pS values of 6.1 and 6.4. It should be noted that for carboxyl-containing substances whose acid forms are water soluble, the pKa and pS values are quite similar.

TABLE 1

| | | | | Ratio of Percent |
|---|---|---|---|---|
| | | Percent in Acid Form | | in Acid Form at |
| Acid | pKa/pS | at pH 6.4 | at pH 7.4 | pH 6.4/at pH 7.4 |
| Propionic Acid | 4.87 | 2.9% | 0.3% | 9.7 |
| Chlorambucil | 5.80 | 20.1% | 2.4% | 8.2 |
| Advanced | 6.10 | 33.4% | 4.8% | 7.0 |
| pH-switch1 | | | | |
| Advanced | 6.40 | 50.0% | 9.1% | 5.5 |
| pH-switch2 | | (relates to | | (relates to |
| | | efficacy) | | therapeutic index) |

From Table 1 it can be seen that for such substances a higher pKa or pS value correlates with a higher fraction of that substance existing in the acid form in acidic areas of a tumor (pH 6.4). For the case of an onco tool, this greater percent in the acid form is expected to correlate with greater retention in acidic areas of a tumor, which translates to a stronger diagnostic signal or greater therapeutic efficacy.

However, it is also clear from that table that a higher pKa or pS gives a corresponding increase in the amount of substance existing in the acid form at pH 7.4, which will cause increased retention in normal tissues. In the diagnostic application this would be expected to generate a greater background signal from normal tissues surrounding a tumor, perhaps so much so that it could swamp out the signal from the tumor. In the therapeutic application the increased retention in normal tissues would be expected to reduce the therapeutic index (i.e., activity against tumor/activity again normal tissues) to such a low level that the therapy causes great harm to or death of the patient.

The problem one faces then is that if the pS of the carboxylic acid moiety of the pH-switch of an onco tool is fairly low, such as with a conventional carboxylic acid, then the diagnostic signal or therapeutic efficacy for that onco tool is expected to be unduly low. Conversely, if the pS is substantially higher, as can be achieved with advanced pH-switches, then the background to the diagnostic signal could easily become so high as to swamp out the signal from the tumor, or the therapeutic index could become impractically low, resulting in great harm to the patient.

This efficacy/specificity challenge is particularly forbidding in attempts to exploit the acidity in tumors because one only has about a 1.0 pH unit differential between tumor and normal tissues. In this regard, based on a simple application of the Henderson-Hasselbalch equation, such a modest pH differential between tumor and normal tissues has led some to conclude that there is an upper limit of only about 10 on the maximum therapeutic index which might be achievable with a therapeutic agent designed to exploit the pH differential between normal tissues and acidic areas of tumors.

High-Specificity Onco Tools

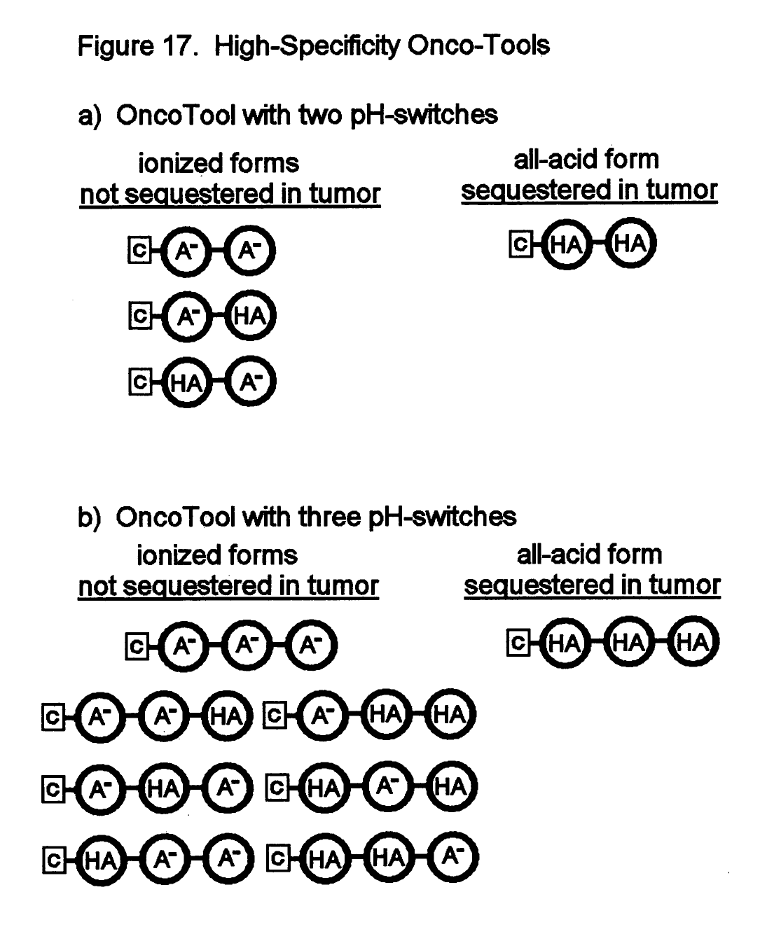

Contrary to conventional wisdom's expectation of a quite modest upper limit on the therapeutic index for agents designed to exploit the acidity of tumors, recently the inventor devised a practical structural design strategy for substantially increasing the specificity of onco tools for acidic areas of tumors. In essence, the strategy is to design onco tools which contain two or more pH-switches, where the carboxyl of each pH-switch is sufficiently distant (greater than about 5 angstroms) from the carboxyl of its neighboring pH-switch that ionization of one does not significantly affect the ionization of its neighbor. Conversely, the neighboring pH-switches of such oligomeric onco tools must be in sufficiently close proximity that all (or for higher oligomers, at least most) of the component pH-switches must be in the acid form in order for that onco tool to be sequestered in acidic areas of tumors. This is illustrated in FIG. 17a for an onco tool containing two pH-switch components, and in FIG. 17b for an onco tool containing three pH-switch components.

In this context, it should be noted that the pH-switch peptides, such as shown in FIG. 4a, can be envisioned as a linear chain of multiple individual pH-switches, where each such pH-switch component comprises one glutamic acid plus its neighboring lipophilic amino acids. To a large extent it appears to be the ratio of the glutamic acids to the lipophilic amino acids in such peptides which determines their respective pS values. It is noteworthy that these pS values are substantially higher than one would expect for peptides containing multiple glutamic acids. Most probably this substantial increase in these pS values over the expected is a consequence of the hydrophilicity/lipophilicity effect detailed in FIGS. 8 and 9 and Example 1. In this regard, when the peptides in FIG. 5 are in their alpha-helical configuration, the ratio of non-polar hydrogens to H-bonding sites exposed to the solvent ranges from a low of 3.6 for core repeating sequence vi, to a high of 5.0 for core repeating sequence i. It is noteworthy that these ratio values are in about the same range as the ratios which have been found to be desirable for advanced pH-switches, such as illustrated in FIG. 16.

Table 2 shows calculated values as in Table 1, but now includes onco tools containing one, two, and three advanced pH-switches, and the calculated values are for that fraction of an onco tool wherein all of its pH-switches are in the acid form at the selected pH. In these calculations the Henderson-Hasselbalch equation was used to calculate the relative concentration of salt and acid forms at a given pH for a substance having acid moieties with a specified pKa or pS value. Then a binomial expansion was used to calculate the concentration of the all-acid form for an onco tool containing 2 or 3 pH-switch components.

TABLE 2

| | | | | Ratio of Percent |
|---|---|---|---|---|
| | | | | in Acid Form at |
| | | Percent in Acid Form | | pH 6.4/at |
| Acid | pKa/pS | at pH 6.4 | at pH 7.4 | pH 7.4 |
| Propionic Acid | 4.87 | 2.9% | 0.3% | 9.7 |
| Chlorambucil | 5.80 | 20.1% | 2.4% | 8.2 |
| Onco Tool 1 | 6.10 | | | |
| One pH-switch | | 33.4% | 4.8% | 7.0 |
| Two pH-switches | | 11.2% | 0.2% | 49.0 |
| Three pH-switches | | 3.7% | 0.01% | 342.3 |
| Onco Tool 2 | 6.40 | | | |
| One pH-switch | 50.0% | 9.1% | 5.5 | |
| Two pH-switches | 25.0% | 0.8% | 30.3 | |
| Three pH-switches | 12.5% | 0.08% | 166.4 | |
| | | (relates to | | (relates to |
| | | efficacy) | | therapeutic index) |

These calculated values suggest that onco tools containing two or more advanced pH-switch components may provide substantially improved specificities for tumors, relative to onco tools containing just a single pH-switch component. The results also suggest that at pH 6.4 enough of the oligomeric onco tools should be in their all-acid form to achieve reasonably efficient sequestering in acidic areas of tumors.

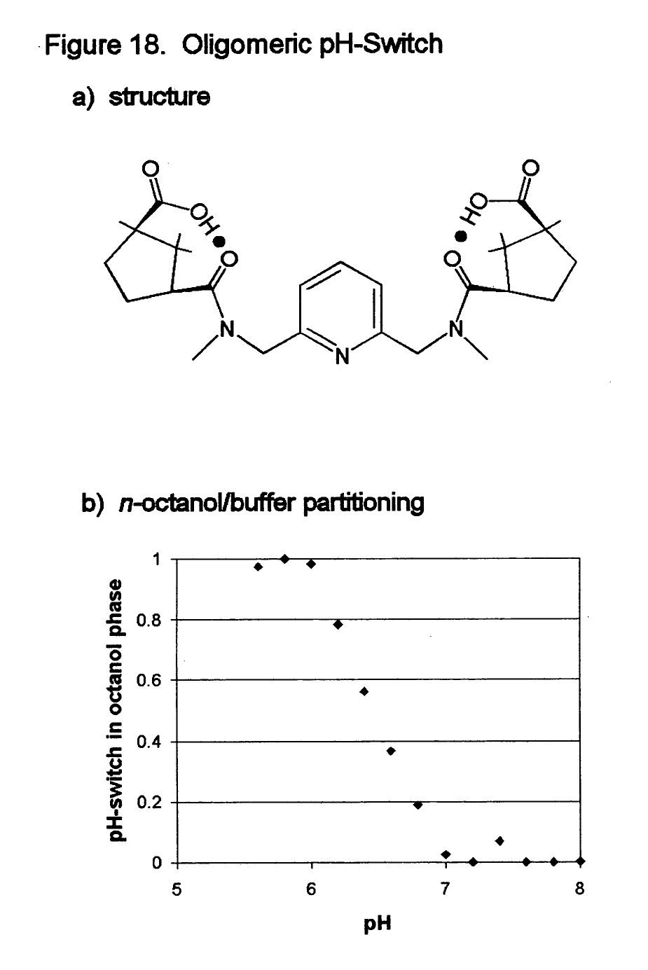

FIG. 18a shows a representative composition containing two advanced pH-switch components. After synthesis, this composition was partitioned between n-octanol and aqueous buffer, and the results are plotted in FIG. 18b. These results indicate that at pH 6.4 about half of this composition partitioned into the octanol phase, while at pH 7.0 only about 2% partitioned into the octanol, and at pH 7.2 none of the composition was detected in the octanol phase.

As suggested by the data in Table 2 regarding onco tools with multiple pH-switches, these partitioning results demonstrate that the majority of a composition containing two advanced pH-switch components can exist in its all-acid form (i.e., octanol-soluble form) at the pH which is present in acidic areas of tumors, and then that composition can switch virtually completely to its salt form (i.e., buffer-soluble) at the pH present in normal tissues. This provides experimental evidence for the value of incorporating multiple pH-switch components into an onco tool as a means for greatly increasing the onco tool's specificity for acidic areas of tumors.

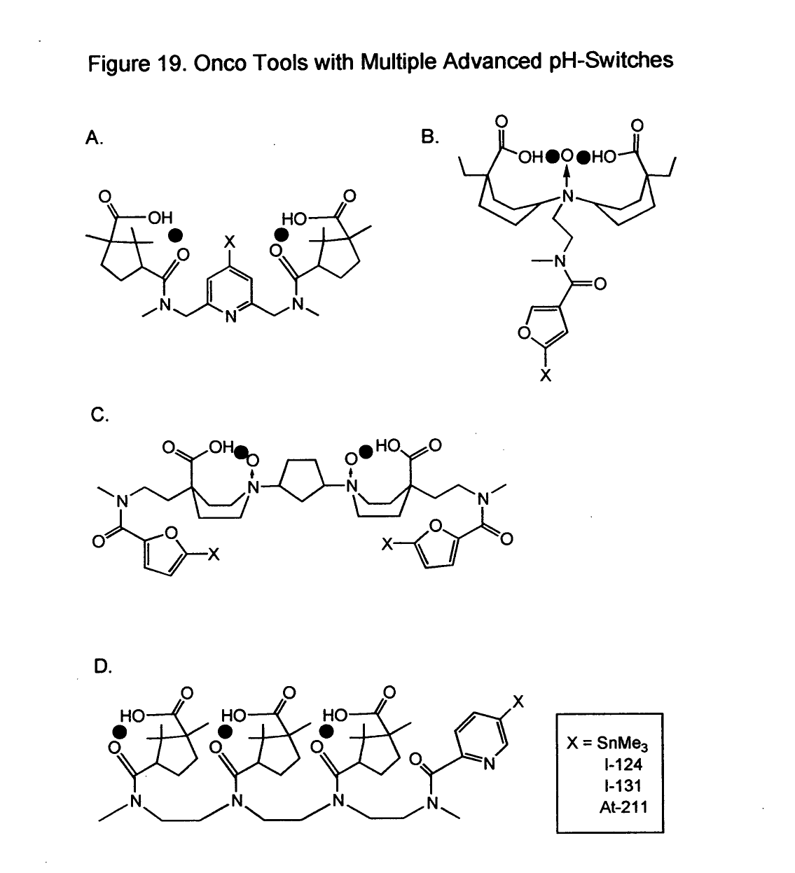

FIG. 19 illustrates a variety of ways two or more advanced pH-switch components plus a suitable cargo component can be joined to form a high-specificity onco tool.

D. Improved Therapeutic Strategy

Therapeutic onco tools were initially devised solely as a means for killing treatment-resistant cells in acidic areas of tumors. Because onco tools are designed to be sequestered only in acidic areas of tumors, it was expected that such onco tools would be used in conjunction with more conventional cancer therapies, such as radiation therapy or chemotherapy, in order to also kill the treatment-sensitive fast-dividing tumor cells in onco tool-free areas of tumors having better oxygenation and higher pH (see pending patent applications by applicant, patent application Ser. Nos. 11/069,849, pending; and 11/069,387, allowed but not yet published or issued).

More recently the inventor has devised an improved therapeutic strategy wherein onco tools alone can be used to destroy the entire tumor, thereby obviating the need for co-treatment with more toxic conventional cancer therapies. This combination therapeutic onco tool strategy entails using two onco tools effective to kill cells. One of the onco tools contains an alpha-emitting radioisotope which serves to kill treatment-resistant quiescent cells in very close proximity to onco tool sequestered in acidic areas of the tumor. The other onco tool contains a beta-emitting radioisotope, which serves to kill from a distance the treatment-sensitive fast-dividing tumor cells in less-acidic areas of the tumor, where such cells may be as much as about 50 to several hundred micrometers from onco tool sequestered in acidic areas of the tumor.

The most preferred alpha-emitting radioisotope for such combination onco tool therapy is Astatine-211, with Actinium-225 also having favorable properties.

While there are a number of beta-emitting radioisotopes which can be used in such a combination onco tool therapy, Iodine-131 is particularly preferred. Other beta-emitting radioisotopes with favorable properties include Bromine-82, Rhenium-186 and Yttrium-90.

Compared to treatment where onco tools are used only to kill the quiescent cells in acidic areas of tumors, and are combined with conventional cancer therapies effective to kill the fast-dividing tumor cells in other areas of tumors having high pH, it is expected that this new combination therapeutic onco tool strategy wherein onco tools alone are used to kill the entire tumor, will afford a simpler, less costly, far less toxic, and far more effective treatment.

E. Design, Optimization, and Preparation of pH-Switches and Onco Tools

1. Preparation of pH-Switch Peptides

Peptides of the present invention, such as peptides containing the core repeating sequences shown in FIG. 5a, can be synthesized on 1% cross-linked polystyrene resin of the Wang type or the hydroxymethyl type using an automated peptide synthesizer supplied with fluorenylmethoxycarbonyl-protected/pentafluourophenyl ester-activated amino acids. Typically, all the amino acids are of the unnatural D chirality, in order to give peptides resistant to peptidases and proteases present in biological systems. The C-terminal amino acid of the peptide is typically linked to the synthesis resin via an ester link cleavable with a primary amine. In many cases the C-terminal amino acid is an alanine, which is then followed by a core sequence comprising multiple repeats of a selected short sequence suitable for the pH-switch. Preferred sequences suitable for pH-switches are shown in FIG. 5a. Reagents and methods for preparing such peptides are well known in the peptide synthesis art and are available from and detailed in the NovaBiochem Handbook and Catalog, 2000.

Preferred pH-switch peptides disclosed in the present application include the core repeating sequences shown in FIG. 5a. Processing of such peptides and their use in preparing representative onco tools containing specific pH-switch peptides are described in sections 4 and 5 below.

2. Design and Optimization of Advanced pH-Switches

An effective approach to designing an advanced pH-switch is to start with a molecular model of a 4-membered, 5-membered, or 6-membered aliphatic ring, such as illustrated in FIG. 12a. The ring, particularly if it is a six-membered ring, may also have one or more additional groups which serve to further limit the conformational freedom of the ring or H-bonding elements attached to the ring, or to favor the ring conformation which positions the H-bonding elements optimally for formation of the desired internal H-bond. For instance, a 6-membered ring with a carboxyl at the 1 position and an H-bond acceptor moiety at or linked to the 4 position, may have an additional 2-carbon bridge between the 2 and 5 carbons of the ring in order to lock the ring into the twisted-boat conformation—which is the optimal conformation for forming the desired internal H-bond between the H-bond donor and acceptor moieties. One next adds a carboxylic acid moiety which will serve as the H-bond donor, and then adds a suitably-positioned H-bond acceptor moiety, generally a nitrogen or oxygen, which must have a structure such that it cannot also serve as an H-bond donor. While computer-based modeling is suitable for this initial design process, the inventor finds that plastic CPK molecular models are fast, easy, and provide a particularly good appreciation of the stereochemistry of such structures in all three dimensions.

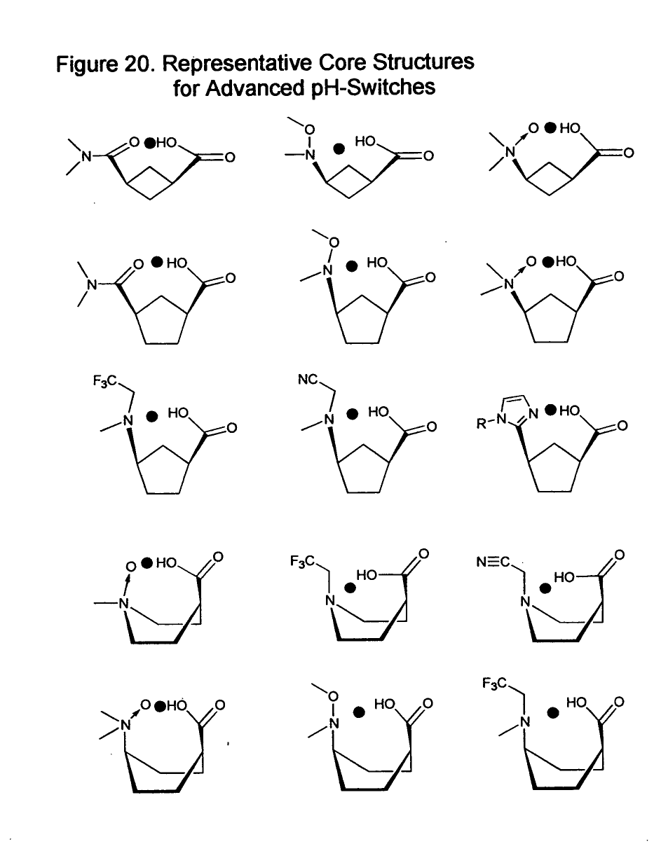

While there are a number of possibilities for the H-bond acceptor moiety, the carbonyl oxygen of an amide, such as illustrated in FIG. 11a, has been found to be effective and readily accessible synthetically. Alternatively, H-bond acceptor moieties which are suitable for forming a low-barrier H-bond with the carboxylic acid donor, such as illustrated in FIG. 15a, are particularly desirable. FIG. 20 illustrates a variety of representative core structures suitable as starting points for advanced pH-switches.

Once a core structure has been selected, one generally needs to further optimize the structure so that its anionic salt form is sufficiently hydrophilic that is has little or no affinity for tissues, while its non-ionic acid form is sufficiently lipophilic that it is effectively sequestered in tissues. A method for such structural optimization has been described earlier herein and is illustrated in FIG. 16, and entails adding various alkyl groups to suitable sites on the core structure, and then carrying out initial testing in a simple titration assay in aqueous solution, followed by more advanced testing, such as partitioning and membrane binding, on the more promising structures.

After promising structures have been identified, one must also incorporate into such structures a means for linking them into an onco tool, either via linkage to a pH-switch peptide component or another advanced pH-switch component, or via linkage to a cargo component. Ultimately, the most promising of these onco tools must be tested in tumor-bearing animals.

3. Design of Cargo Component

Cargo Component for Large Onco Tools

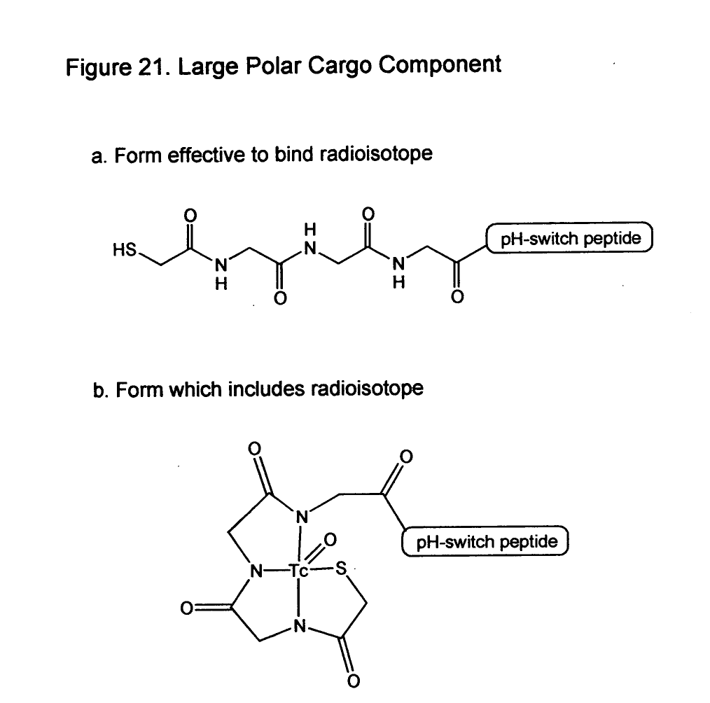

For onco tools containing a relatively large pH-switch peptide component (typically about 1,800 to 4,000 daltons) one can use a relatively polar cargo component suitable for rapidly and efficiently chelating a metal oxide-type radioisotope, such as Technetium-99 or Rhenium-186. A representative cargo component of this type known as MAG3, which is well known in the nuclear medicine field, is shown in FIG. 21. FIG. 21a shows that cargo component in its form which is effective to bind a metal oxide-type radioisotope. It is the onco tool with this “effective-to-bind” form of the cargo component which will be synthesized, stored, shipped to the user, and stored by the user until needed. FIG. 21b shows that cargo component with a bound radioisotope. Generally just before introduction of the onco tool into the patient the radioisotope will be added to the onco tool form shown in FIG. 21a, by established methods known in the nuclear medicine field, and this will generate the onco tool form wherein the cargo component now includes the bound radioisotope, as shown in FIG. 21b.

Thus, the onco tool is produced, shipped, and stored in a form wherein the cargo component is effective to bind a radioisotope, and then just before injection into the patient that form of the onco tool is contacted with an appropriate radioisotope (typically prepared in a nuclear reactor or a cyclotron) to generate the final form of the onco tool wherein the cargo component now includes a radioisotope. It is this final radioisotope-containing form of the onco tool which is delivered into the patient, and which will be sequestered into acidic areas of tumors if such tumors are present in the patient.

Cargo Component for Small Onco Tools

In contrast to the case for relatively large peptide-containing onco tools, for relatively small onco tools containing no peptide component and only one or a few advanced pH-switch components (typically about 400 to 1,200 daltons) it is desirable to use a relatively small cargo component which is neither strongly lipophilic nor strongly hydrophilic. This is because a strongly lipophilic cargo component can cause undue binding of the small onco tool to normal tissues (pH 7.4), while a strongly hydrophilic cargo component can disfavor tissue binding of the small onco tool in acidic areas of tumors. Further, it is essential that said cargo component be effective to readily and efficiently bind its radioisotope with minimal manipulation, and that the bound radioisotope not be readily cleaved off the cargo component in vivo.

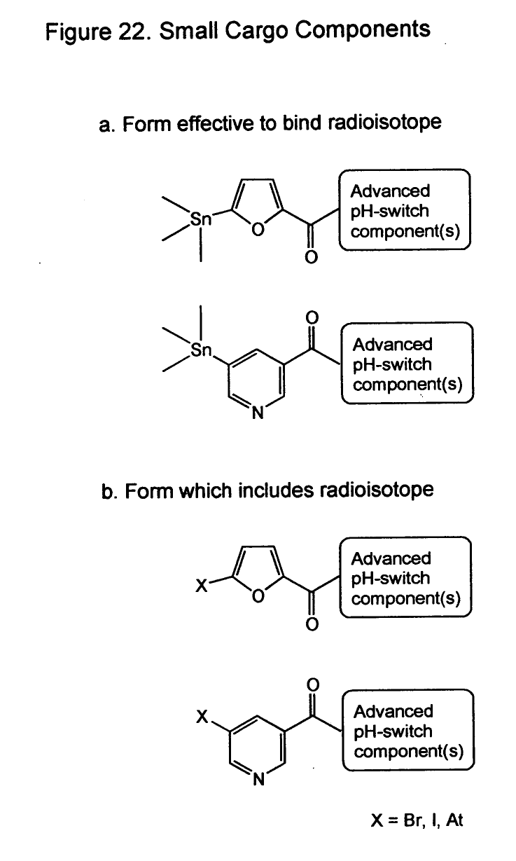

In light of these requirements, radiohalogens, selected from Bromine, Iodine, and Astatine, are the preferred radioisotope type, and small moderately-water-soluble aromatic rings are preferred for binding such radioisotopes. FIG. 22a shows two such representative cargo components in a form effective for readily and efficiently binding a radiohalogen, and FIG. 22b shows those cargo components after the radiohalogen is bound. In this regard, a tributyl tin moiety is readily displaced by Bromine and Iodine, while a trimethyl tin moiety is readily displaced by Bromine, Iodine, and Astatine (Zalutsky et al., Proc. Natl. Acad. Sci. USA. vol 86 pages 7149-7153 (1989)).

It should be noted that the smaller cargo components in FIG. 22 are also entirely suitable for use with the relatively large onco tools containing a pH-switch peptide component.

4. Preparation of Onco Tools Containing Improved pH-Switch Peptides

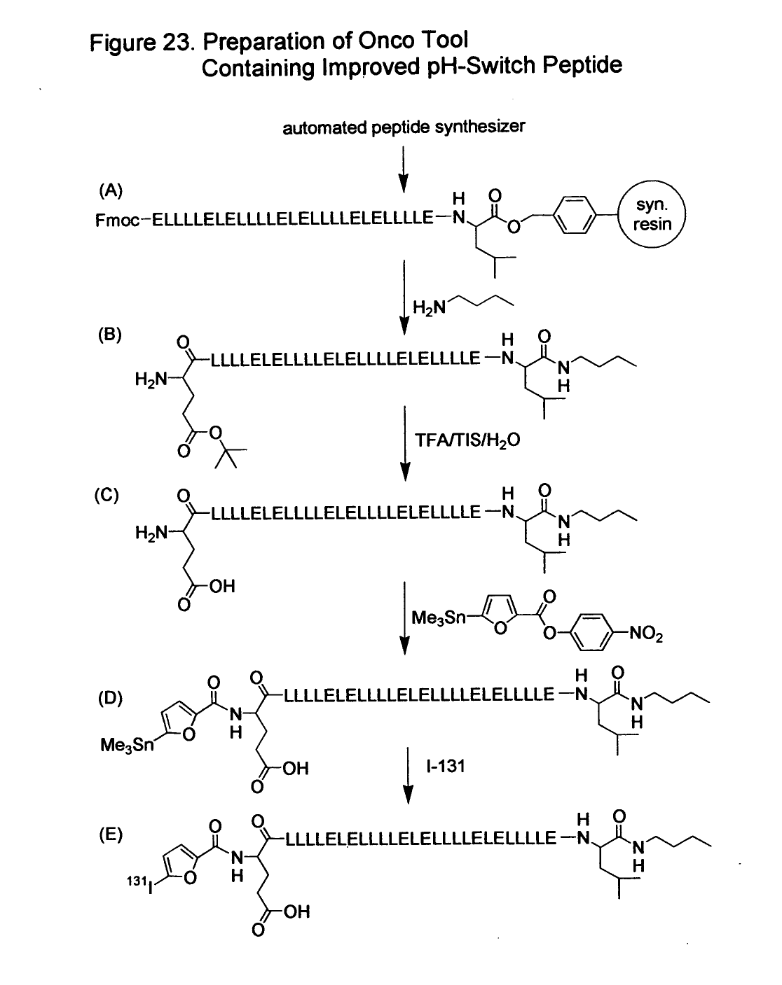

FIG. 23 shows the steps in preparing a representative onco tool containing an improved pH-switch peptide component of the invention. The peptide contains the amino acid sequence shown in FIG. 4a and also shown in FIG. 5a, repeating sequence type iv, n=4.

Structure D in FIG. 23 is the form of the onco tool which is effective to bind a radiohalogen. It is the structure D form that is prepared and is suitable for storage and shipment to the end user, who may also store it. At a desired time the end user then contacts the structure D onco tool with I-131 (or other radiohalogen of choice) to generate final radioisotope-containing structure E onco tool that is injected into the patient. Procedures for displacing the trimethyl tin moiety with Iodine are detailed in Zalutsky et al, referenced in the earlier section describing the design of the cargo component.

5. Preparation of Onco Tools Containing a pH-Switch Peptide and an Advanced pH-Switch

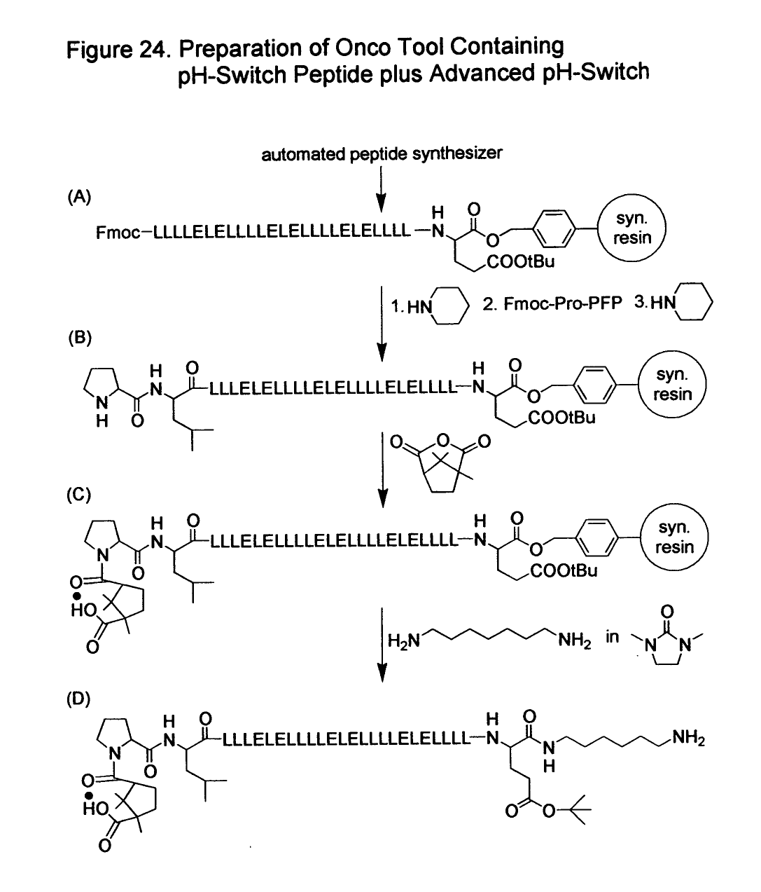

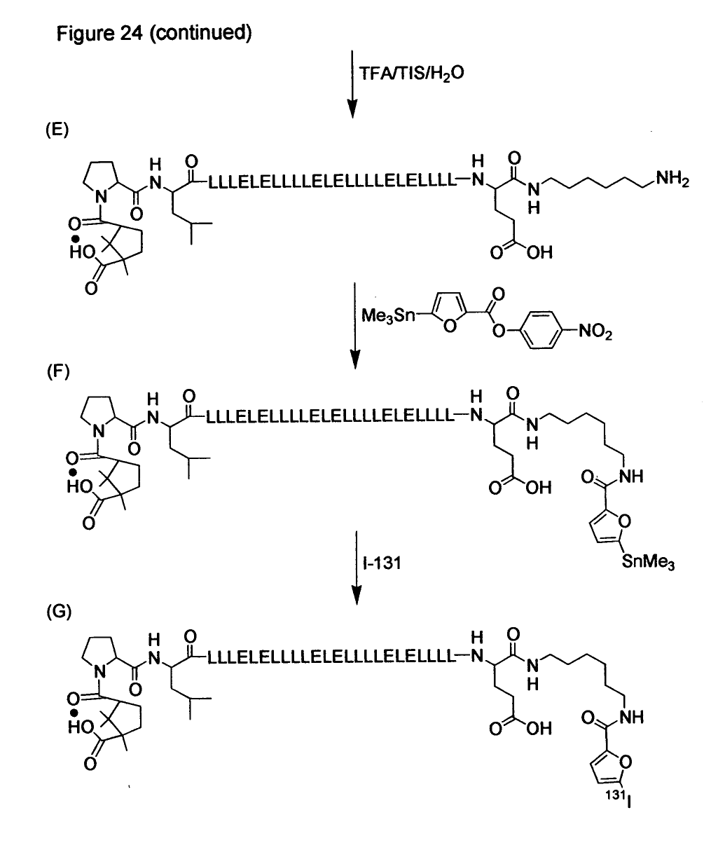

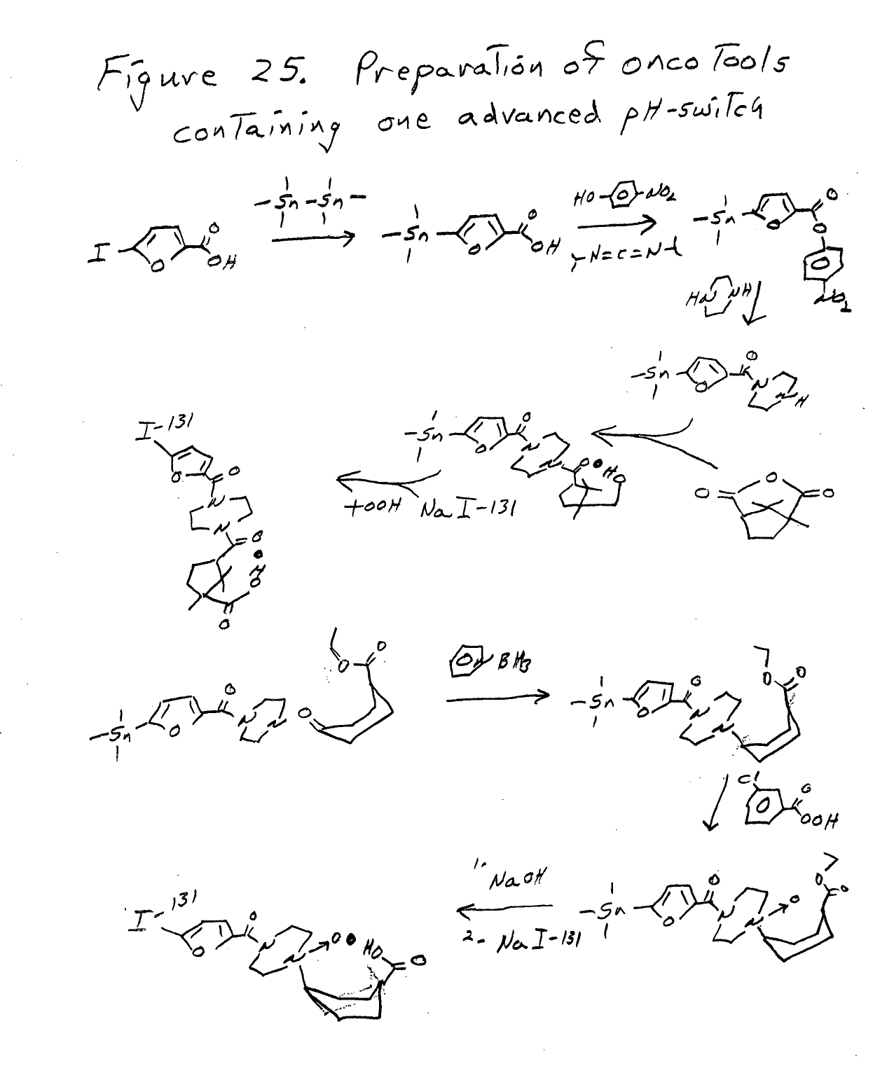

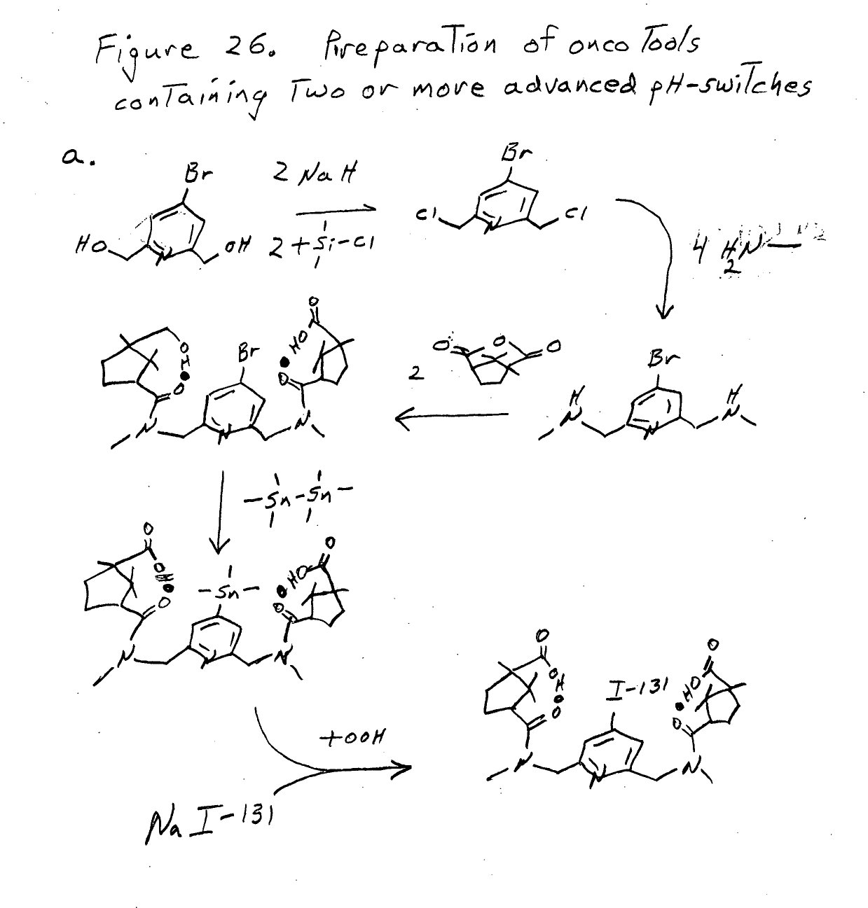

FIG. 24 shows the steps in preparing a representative onco tool containing a pH-switch peptide component having a sequence similar to that shown in FIG. 4a, but without the N-terminal glutamic acid and without the C-terminal leucine. Linked to the end of that pH-switch peptide is an advanced pH-switch having a structure of the type shown in FIGS. 11a, 14b, and 16a.

Structure F in FIG. 24 is the form of the onco tool which is effective to bind a radiohalogen. It is the structure F form that is prepared and is suitable for storage and shipment to the end user, who may also store it. At a desired time the end user then contacts the structure F onco tool with I-131 (or other radiohalogen of choice) to generate the final radioisotope-containing structure G onco tool that is injected into the patient. Procedures for displacing the trimethyl tin moiety with Iodine are detailed in Zalutsky et al, referenced in the earlier section describing the design of the cargo component.

6. Preparation of Onco Tools Containing One Advanced pH-Switch

FIG. 25 shows several synthetic routes for preparing representative onco tools, each containing a single advanced pH-switch component.

7. Preparation of Onco Tools Containing Two or More Advanced pH-Switches

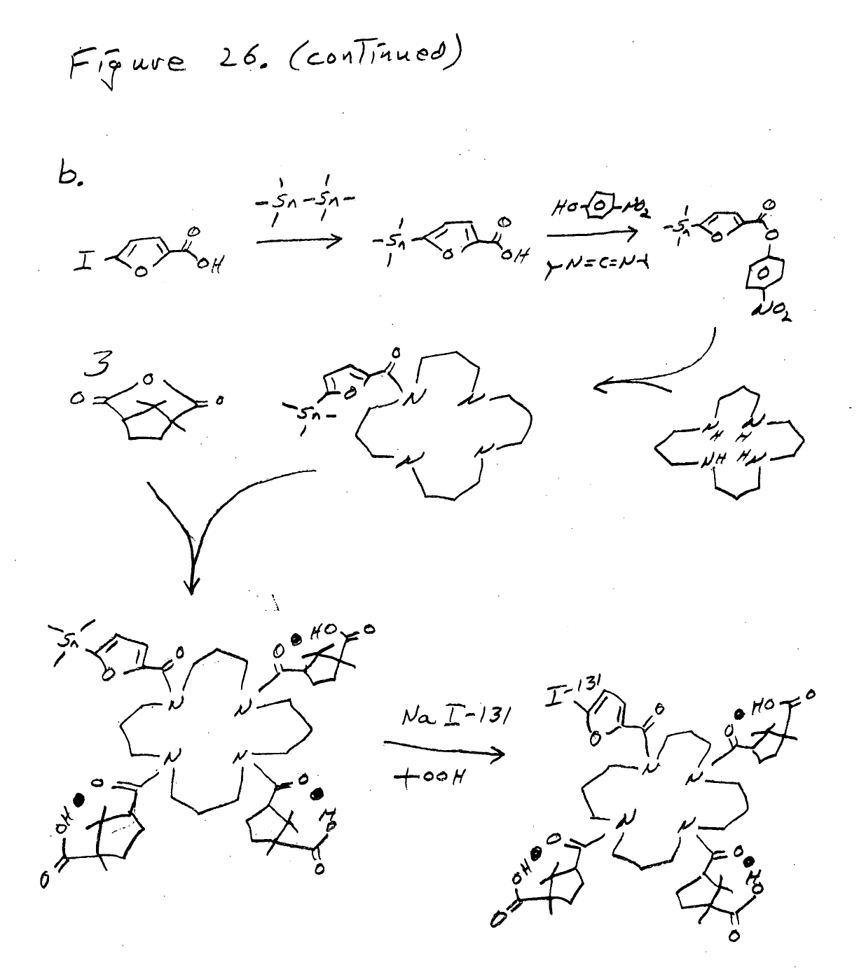

FIG. 26 shows several synthetic routes for preparing representative onco tools, each containing two or more advanced pH-switch components.

G. Testing pH-Switches and Onco Tools

1. Titration Assay

Typically new structures that may be suitable as advanced pH-switch components are first assessed for their pS value. This entails preparing the sodium salt form of the structure and dissolving it at a concentration of 20 milliMolar in 15 ml of 0.15 Molar NaCl (deareated to remove CO2). A small magnetic stir bar is added and the pH of the solution adjusted to about 9 with a small amount of 5 M NaOH. While stirring, 5 microLiter portions of 5 M HCl are added and the pH is recorded at 1 minute after addition of each portion. A plot of pH versus volume of added HCl affords the pS value.

2. Precipitation Assay

A precipitation assay is a useful alternative to the titration assay and requires far less material. The precipitation assay is appropriate when the pH-switch or onco tool contains a moiety readily detectable in the UV absorbance range, such as is the case for the structure in FIG. 18, and the cargo components in FIG. 22. In this assay one first prepares a series of buffers having pH values ranging from 5.4 to 8.0 in 0.2 pH increments. Each buffer is 0.14 M in NaCl and 0.015 M in buffer. Preferred buffers are:

a) 2-(Morpholino)ethanesulfonic acid (MES, pKa 6.1);

b) N-(2-Acetamido)-2-aminoethanesulfonic acid (ACES, pKa 6.8); and,

c) 4-(2-Hydroxyethyl)piperazine-1-ethanesulfonic acid (HEPES, pKa 7.5).

A 1 ml portion of each buffer prep is placed in a 1.5 ml centrifuge tube and 10 microliters of sample solution is added. Typically the stock sample solution is the sodium salt form of the test compound in water at a concentration sufficient go give an absorbance value of about 100, so that when 10 microliters is diluted into the 1 ml of buffer the absorbance will be about 1.0-minus any quantity which has precipitated (which generally occurs in the lower-pH buffer solutions). Each vial is then capped, shaken well, allowed to sit at room temperature for 30 minutes, and then centrifuged in a microcentrifuge at 10,000 rpm for 10 minutes. Supernatants are then assessed in a UV/Vis spectrophotometer and the absorbance values plotted as a function of pH.

Onco Tools, particularly ones containing an improved pH-switch peptide with an advanced pH-switch on one terminus, can show a quite sharp pH-dependent switch between soluble (salt) and insoluble (acid) forms, with about 90% of the transition occurring within a range of about 0.5 pH unit for the most preferred structures closely related to structure G of FIG. 24.

3. Octanol/Buffer Partitioning Assay

Like the precipitation assay above, a partitioning assay is most practical when the substance to be tested contains a moiety readily detectable in the UV absorbance range. In this assay a 0.5 ml portion of each buffer preparation, as described in the previous section, is placed in a 1.5 ml centrifuge tube and 10 microliter of the stock sample solution, as described in the previous section, is added to the buffer solution. Next, 0.5 ml of n-octanol is added to each vial, the vials capped and shaken vigorously for 2 minutes, and then centrifuged to separate the phases.

A 0.3 ml portion of each octanol top phase is added to 0.5 ml of isopropanol and the absorbance measured, and these absorbance values for all samples plotted as a function of the pH of the buffer phase. Likewise, a 0.3 ml portion of each lower buffer phase is added to 0.5 ml of water and the absorbance measured, and the absorbance values for all samples plotted as a function of the pH of the buffer phase.

4. Binding to Isolated Cell Membranes

Red cell ghosts are prepared from fresh blood by the standard osmotic lysis and repeated centrifugal washes in normal saline. About 0.5 ml of packed red cell ghosts are resuspended in 1.5 ml of 0.15 M NaCl/0.01 M sodium azide to give a stock solution of isolated cell membranes. Each time before using this stock membrane suspension swirl it briefly for a few seconds. This stock membrane suspension can be used and stored at 4 deg. C. for two or three days.

As with the precipitation and partitioning assays, membrane binding assays are most suitable when the substance to be assayed has a substantial absorbance in the visable or UV range.

A typical membrane binding assay is carried out much like the precipitation assay. Specifically, a 1.0 ml portion of each buffer preparation, prepared as for the precipitation assay above, is placed in a 1.5 ml centrifuge tube and 10 microliters of sample solution is added. Typically the stock sample solution is the sodium salt form of the test compound in water at a concentration sufficient to give an absorbance value of about 100, so that when 10 microliters is diluted into the 1 ml of buffer the absorbance will be about 1.0. Next, 75 microliters of the stock membrane suspension described above, is added, the tube is capped and swirled briefly, and then all the tubes incubated at 37 deg. C. for 30 minutes. The vials are centrifuged in a microcentrifuge at 10,000 rpm for 10 minutes to pellet the red cell ghosts, and the supernatants then assessed in a UV/Vis spectrophotometer. Finally, the absorbance values are plotted as a function of pH.

While in principle it is difficult to distinguish between simple precipitation and membrane binding in the above assay, in practice the membranes in the assay can have a significant impact for some pH-switches, but little or no impact for other pH-switches.

5. Assessment of Onco Tool Sequestering in Tumor-Bearing Mice

The decisive test for an onco tool is to react it with a suitable radioisotope which generates a readily quantifiable signal, such as I-131, and then inject that onco tool into tumor-bearing mice. After allowing adequate time for washout of unsequestered onco tool (on the order of about 5 to 20 hours), one can then scan the mouse with a gamma camera to identify where and roughly how much onco tool is retained in the various major organs. Alternatively, more precise results can be obtained by dissecting and counting each major organ and any obvious tumors. Procedures for such testing in live animals are well known in the nuclear medicine field, and particularly in the sub-field of radioimmunotherapy.

H. Preferred Methods for Detecting and Treating Tumors

It should be noted that any given onco tool containing a cargo component in its “effective to bind” form, such as shown in FIGS. 21a and 22a, can generally be used either for detection of tumors, or for treating tumors. This dual potential is because it is the specific radioisotope which determines the function the onco tool will carry out.

For example, if the cargo component of the onco tool constitutes the MAG3 group shown in FIG. 21a, and that onco tool is reacted with the gamma-emitting radioisotope, Technetium-99. then the resultant radioisotope-containing onco tool will function for the detection of tumors. Conversely, if that onco tool is instead reacted with the beta-emitting radioisotope, Rhenium-186, then the resultant radioisotope-containing onco tool will function for killing cells.

As another example, if the cargo component of the onco tool contains the trimethyl tin moiety shown in FIG. 22a, and that onco tool is reacted with the positron-emitting radioisotope, Iodine-124, then the resultant radioisotope-containing onco tool will function for the detection of tumors. Conversely, if that onco tool is instead reacted with the beta-emitting radioisotope, Iodine-131 or the alpha-emitting radioisotope, Astatine-211, then the resultant radioisotope-containing onco tool can function for killing cells. However, because both of these particular radioisotopes also have emissions detectable at a distance, they can also function for detection of tumors.

Thus, in general onco tools in their stable “effective-to-bind” form suitable for shipping and storage can be converted by the end used, by virtue of that user's choice of radioisotope which is reacted with that onco tool, to a final form suitable for detection of tumors, or to a final form suitable for treatment of tumors, or to a final form which is suitable for detection and treatment of tumors.

In regard to using an onco tool in veterinary or human medicine, be it for detection or for treatment of tumors, there are a number of points which should be considered.

First, if the treated subject has urine which is acidic (commonly the case) then most or all onco tools are expected to become sequestered in cells lining the proximal tubules of the kidneys. In a diagnostic mode this could obscure a signal from a tumor located in or near the kidney. In a therapeutic mode this could destroy the kidneys. Luckily, there are a number of safe over-the-counter drugs, such as Acetazolamide, which can be used to raise and maintain the urine at a pH greater than 7.5. While this precaution is probably adequate for the case of onco tools which contain only the advanced pH-switch components, for the case of onco tools which contain pH-switch peptides there is greater concern because the kidney has an aggressive reuptake mechanism for peptides.

In light of their lack of basic amino acids, and particularly for pH-switch peptides prepared from D-amino acids (the un-natural chirality), it is unlikely reuptake will be a problem as long as the urine is kept at a pH above 7.5, but it needs to be carefully checked for. While a re-uptake problem is far less likely for onco tools containing only advanced pH-switch components, they must also be checked carefully.

There are measures which can be taken to block reuptake of peptides, if it turns out to be a problem for a given onco tool, but probably the safer approach is to identify a different onco tool which does not suffer from reuptake. This is particularly the case for therapeutic applications where significant re-uptake by the kidneys could destroy them. As noted above, onco tools containing no pH-switch peptide component, but one or a few advanced pH-switch components are most likely to be free of re-uptake problems—as long as the urine is maintained at a pH above about 7.4 through the use of a suitable drug for this purpose.

Second, it is likely that the lower the pH is in acidic areas of tumors the more efficiently onco tools will be sequestered in such areas. In this regard, there are several established methods that can be employed to further reduce the pH in tumors, without a corresponding reduction of the pH in normal tissues. One method is to introduce a substantial concentration of glucose into the subject. This has been shown to lead to a reduction of about 0.3 pH in the acidic areas of tumors (Kozin et al., Cancer Research Vol. 61, pages 4740-4743 (2001)). Alternatively, vasodilators, which are very safe and widely used drugs for reducing blood pressure, have also been shown to be effective for selectively reducing the pH in acidic areas of tumors—again by about 0.3 pH unit. Other drugs for further selectively reducing the pH in tumors are mitochondrial inhibitors.

### EXAMPLES

### Example 1

The sodium salts of propionic acid and octanoic acid in water were titrated with 5 M HCl. The mid-point of the transition between salt and acid (pKa value) was found to be an expected 4.83 for propionic acid, but a surprisingly-high 5.5 for octanoic acid. The first derivative of the titration curve was symmetrical for the propionic acid, but highly unsymmetrical for the octanoic acid. In addition, droplets of octanoic acid came out of solution during the course of the octanoic titration.

In contrast, when the salts of these same two acids were titrated in methanol/water, 1:1 by vol., their mid-points of transition were virtually identical and the first derivative of their titration curves were now both symmetrical. Finally, in the titration in methanol/water no octanoic acid came out of solution.

These results suggest that the surprisingly high mid-point in the titration seen for octanoic acid in aqueous solution was due simply to the solubility effects illustrated in FIGS. 8 and 9. The results further suggest that carrying out the titrations in methanol/water avoids these solubility effects on the titration curves.

### Example 2

The sodium salts of compounds shown in FIG. 11a (designed to form internal acid-specific H-bond) and 11b (designed to form non-acid-specific H-bonds) were titrated in aqueous solution. The first derivative of the titration curve for the compound in FIG. 11b was symmetrical and showed an expected pKa value of 4.82, and there was no apparent insoluble material generated during the titration. In sharp contrast, the first derivative of the titration curve for the compound in FIG. 11a was highly skewed and showed a minimum at the surprisingly high value of 5.8, with the approximate mid-point of the titration at pH 5.6. Further, there was massive precipitate formed with each addition of HCl, starting when the pH reached 5.8.

These dramatic differences for nearly identical compounds suggest that the structure in FIG. 11a is forming the predicted internal acid-specific H-bond, and the structure in FIG. 11b is not forming such an H-bond.

### Example 3

Titration of the 5-membered ring structure of FIG. 12a and the acyclic structure of FIG. 12b showed an expected pKa value of 4.95 for the acyclic structure, but an unexpectedly-high value of 5.6 for the 5-membered ring structure in FIG. 12a—accompanied by massive precipitation when the pH got below about 5.8, as well as skewing of the shape of the titration curve.

When these two compounds were titrated in methanol/water, 1:1 by vol, there was no sign of insolubility and the first derivative plots of the titration curves were now symmetrical. For the acyclic compound shown in FIG. 12b the pH at the mid-point of the titration was 5.8, while the pH at the mid-point of the titration was a substantially higher 6.45 for the structure in FIG. 12a.

Thus, even in the absence of skewing of the titration curve due to precipitation of the lipophilic acid form, one sees a substantial 0.65 pH unit increase in the mid-point of the titration curve for the compound in FIG. 12a. The most reasonable explanation for this seems to be that an internal acid-specific H-bond is forming and that that H-bond is driving the equilibrium in favor of the acid form.

### Example 4

A molecular model of structure 13b indicated that an internal H-bond should be strongly favored by the near-perfect geometry of said H-bond and the very limited conformational freedom between the H-bond donor and the H-bond acceptor moieties. In spite of these factors, when the salt of the structure in FIG. 13b was prepared and titrated with HCl in aqueous solution, the titration curve indicates a pKa value below 3.0. This result is compatible with the inductive effects from the amide, where said effects should be readily propagated through the intervening double bond, causing a large reduction in the pKa of the carboxyl, with those effects far surpassing any effect an internal H-bond might have on raising the pKa of the carboxyl.

### Example 5

The two compounds in FIG. 14 were prepared and titrated in methanol/water, 1:1 by vol—in order to preclude insolubility effects impacting the titration process. In these experiments the first derivative of the titration plots were symmetrical and there was no visible sign of precipitation. Thus, the objective of avoiding insolubility effects was met.

What was found is that structure a of FIG. 14 showed a mid-point for the titration curve at a pH of 5.6, while structure b of FIG. 14 showed a significantly higher mid-point for the titration at pH 6.14.

Again, a reasonable explanation for the significantly higher value for structure b is that it is forming an internal acid-specific H-bond that is acting to shift the equilibrium in favor of the acid form.

### Example 6

The methyl ester of isonipocotic acid was alkylated with bromopropane and then the resultant tertiary amine oxidized to the N-oxide with meta-Chlorobenzoylperoxide. Finally, the ester was cleaved with aqueous sodium hydroxide to give structure iii in FIG. 15b. This product was titrated in water. The first derivative of its titration plot shows two minimums, one at about pH 4.0 (which is reasonably close to that expected for the N-oxide moiety), and the other minimum at about pH 5.6, which is substantially higher than would be expected for either the N-oxide moiety or the carboxylic acid moiety. However, in light of the results in the earlier examples herein, it appears likely that this 5.6 value is due to an internal low-barrier H-bond forming between the carboxyl H-bond donor and the N-oxide H-bond acceptor, where that internal H-bond acts to drive the equilibrium toward the acid form.

What is claimed is:

1. An onco tool composition which is selectively sequestered in acidic areas of tumors, the composition includes:
A) an improved pH-switch peptide component that under physiological conditions exists predominantly in an anionic/hydrophilic form at pH 7.4, and converts in substantial part to a non-ionic/lipophilic form at a pH above 6.0, said pH-switch peptide component having the following properties:
i) a length from about 14 to about 50 amino acids;
ii) about 20% to about 34% of the amino acids are glutamic acids;
iii) the glutamic acid side chains are not congregated on a face or an end of the alpha helix, but instead are dispersed around the helical axis and along the length of the helical axis;
iv) at least 90% of the non-acid amino acids of the pH-switch peptide are lipophilic amino acids selected from the group consisting of: leucine, isoleucine, norleucine, and methionine;
v) at least one terminus of the pH-switch peptide is predominantly anionic at pH 7.4, and in substantial part non-ionic at a pH above 6.0 by virtue of having a carboxylic acid-containing group having a pKa value greater than 4.0 and positioned at or within one amino acid from the end of the pH-switch peptide;
vi) substantially all of the amino acids are of the same chirality; and,
B) a cargo component selected from the group consisting of:
i) a cargo component that is effective to bind a radioisotope, and
ii) a cargo component that includes a radioisotope.

2. The onco tool composition of claim 1, wherein substantially all of the amino acids of the pH-switch peptide component are of a D chirality.

3. The onco tool composition of claim 1, where the pH-switch peptide component includes an amino acid sequence selected from the group consisting of:
i) [ELLLL]n, n=3 to 10;
ii) [ELLLLELLL]n, n=2 to 5;
iii) [ELLLLELL]n, n=2 to 6;
iv) [ELLLLEL]n, n=2 to 7;
v) [ELL]n, n=5 to 16; and
vi) [ELLLEL]n, n=3 to 8,
where E is glutamic acid and L is a lipophilic amino acid selected from the group consisting of leucine, isoleucine, norleucine, and methionine.

4. The onco tool composition of claim 1, where the pH-switch peptide component includes an amino acid sequence selected from the group consisting of:
i) [ELLLL]n, n=3 to 10;
ii) [ELLLLELLL]n, n=2 to 5;
iii) [ELLLLELL]n, n=2 to 6;
iv) [ELLLLEL]n, n=2 to 7;
where E is glutamic acid and L is a lipophilic amino acid selected from the group consisting of leucine, isoleucine, norleucine, and methionine.

5. The onco tool composition of claim 1, wherein the carboxylic acid-containing group at the terminus of the pH-switch peptide is separated from the next glutamic acid in the peptide chain by four lipophilic amino acids, L, selected from the group consisting of: leucine, isoleucine, norleucine, and methionine.

6. The onco tool composition of claim 1, wherein the cargo component includes a radioisotope which is selected from the group consisting of:
i) a radioisotope which is effective to report the presence of the onco tool;
ii) a radioisotope which is effective to kill cells; and
iii) a radioisotope which is effective to report the presence of the onco tool and effective to kill cells.

7. The conco tool composition of claim 6, wherein the radioisotope has a half-life of less than 20 days and is selected from the group consisting of:
i) an alpha particle emitter;
ii) a beta particle emitter;
iii) a positron emitter; and
iv) a gamma ray emitter.

8. The onco tool composition of claim 7, wherein the radioisotope is selected from the group consisting of:
i) Astatine-211;
ii) Iodine-131;
iii) Bromine-82;
iv) Rhenium-186;
v) Iodine-123;
vi) Iodine-124; and
vii) Technetium-99.

9. An onco tool composition which is selectively sequestered in acidic areas of tumors, the composition includes:
A) an advanced pH-switch component which under physiological conditions exists predominantly in an anionic/hydrophilic form at pH 7.4, and converts in substantial part to a non-ionic/lipophilic form at a pH above 6.0, said advanced pH-switch component having the following properties:
i) contains an aliphatic ring structure selected from the group consisting of: 4-membered rings, 5-membered rings, and 6-membered rings;
ii) contains a carboxylic acid moiety directly linked to said aliphatic ring structure;
iii) said carboxylic acid moiety is separated from any linked electron-withdrawing group by at least two carbons;
iv) contains an H-bond acceptor moiety selected from the group consisting of:
a) part of said aliphatic ring structure;
b) directly linked to said aliphatic ring structure; and
c) linked through one atom to said aliphatic ring structure;
v) said H-bond acceptor moiety has a structure which cannot serve as an H-bond donor moiety;
vi) said carboxylic acid moiety and said H-bond acceptor moiety are positioned in close proximity and are properly positioned and oriented wherein they can form an internal acid-specific H-bond; and,
B) a cargo component selected from the group consisting of:
a) a cargo component which is effective to bind a radioisotope; and
b) a cargo component which includes a radioisotope.

10. The onco tool composition of claim 9, which also includes a pH-switch peptide component having the following properties:
i) a length of about 14 to about 50 amino acids;
ii) about 20% to about 40% of the amino acids are glutamic acids;
iii) the glutamic acid side chains are dispersed around the helical axis and along the length of the helical axis;
iv) at least 90% of the non-acid amino acids are lipophilic amino acids selected from the group consisting of: leucine, isoleucine, norleucine; and methionine; and,
v) substantially all of the amino acids are of the same chirality.

11. The onco tool composition of claim 10, where the pH-switch peptide component includes an amino acid sequence selected from the group consisting of:
i) [ELLLL]n, n=3 to 10;
ii) [ELLLLELLL]n, n=2 to 5;
iii) [ELLLLELL]n, n=2 to 6;
iv) [ELLLLEL]n, n=2 to 7;
v) [ELL]n, n=5 to 16;
vi) [ELLLEL]n, n=3 to 8;
vii) [ELLLELELL]n n=2 to 5; and
viii) [ELLEL]n n=3 to 10.
where E is glutamic acid and L is a lipophilic amino acid selected from the group consisting of: leucine, isoleucine, norleucine, and methionine.

12. The onco tool composition of claim 9, wherein the cargo component includes a radioisotope which is selected from the group consisting of:
i) a radioisotope effective to report the presence of the onco tool;
ii) a radioisotope effective to kill cells; and
iii) a radioisotope effective to report the presence of the onco tool and effective to kill cells.

13. The onco tool composition of claim 9, which includes a radioisotope having a half-life of less than 20 days and is selected from the group consisting of:
i) an alpha particle emitter;
ii) a beta particle emitter;
iii) a positron emitter; and
iv) a gamma ray emitter.

14. The onco tool composition of claim 9, where the radioisotope is selected from the group consisting of:
i) Astatine-211;
ii) Iodine-131;
iii) Bromine-82;
iv) Rhenium-186;
v) Iodine-123;
vi) Iodine-124; and
vii) Technetium-99.

15. The onco tool composition of claim 9, which includes more than one advanced pH-switch components.

16. A method for detecting in a living subject tumors which contain acidic areas, the method comprising:
(a) providing the onco tool composition of claim 1 which includes a radioisotope effective to report the presence of the onco tool,
(b) introducing said onco tool composition into the subject,
(c) waiting for a period of time of about one to about twenty-four hours to allow clearance of said onco tool composition which has not been sequestered in acidic areas of tumors, and
(d) scanning the subject with equipment effective to detect the position and amount of said onco tool composition remaining in the subject.

17. A method for detecting in a living subject tumors which contain acidic areas, the method comprising
(a) providing the onco tool composition of claim 4 which includes a radioisotope effective to report the presence of the onco tool,
(b) introducing said onco tool composition into the subject,
(c) waiting for a period of time of about one to about twenty-four hours to allow clearance of said onco tool composition which has not been sequestered in acidic areas of tumors, and
(d) scanning the subject with equipment effective to detect the position and amount of said onco tool composition remaining in the subject.

18. A method for detecting in a living subject tumors which contain acidic areas, the method comprising
(a) providing the onco tool composition of claim 5 which includes a radioisotope effective to report the presence of the onco tool,
(b) introducing said onco tool composition into the subject,
(c) waiting for a period of time of about one to about twenty-four hours to allow clearance of said onco tool composition which has not been sequestered in acidic areas of tumors, and
(d) scanning the subject with equipment effective to detect the position and amount of said onco tool composition remaining in the subject.

19. A method for detecting in a living subject tumors which contain acidic areas, the method comprising
(a) providing the onco tool composition of claim 9 which includes a radioisotope effective to report the presence of the onco tool,
(b) introducing said onco tool composition into the subject,
(c) waiting for a period of time of about one to about twenty-four hours to allow clearance of said onco tool composition which has not been sequestered in acidic areas of tumors, and
(d) scanning the subject with equipment effective to detect the position and amount of said onco tool composition remaining in the subject.

20. A method for treating tumors in a patient, the method comprising
(a) providing the onco tool composition of claim 1 which includes a radioisotope effective to kill cells,
(b) introducing said onco tool composition into the patient.

21. A method for treating tumors in a patient, the method comprising
(a) providing the onco tool composition of claim 4 which includes a radioisotope effective to kill cells,
(b) introducing said onco tool composition into the patient.

22. A method for treating tumors in a patient, the method comprising
(a) providing the onco tool composition of claim 5 which includes a radioisotope effective to kill cells,
(b) introducing said onco tool composition into the patient.

23. A method for treating tumors in a patient, the method comprising
(a) providing the onco tool composition of claim 9 which includes a radioisotope effective to kill cells,
(b) introducing said onco tool composition into the patient.

24. A method for treating tumors in a patient, the method comprising
(a) providing the onco tool composition of claim 1 which includes a radioisotope which emits an alpha particle,
(b) providing the onco tool composition of claim 1 which includes a radioisotope which emits a beta particle,
(c) introducing both onco tool compositions into the patient.

25. A method for treating tumors in a patient, the method comprising
(a) providing the onco tool composition of claim 4 which includes a radioisotope which emits an alpha particle,
(b) providing the onco tool composition of claim 4 which includes a radioisotope which emits a beta particle,
(c) introducing both onco tool compositions into the patient.

26. A method for treating tumors in a patient, the method comprising
(a) providing the onco tool composition of claim 5 which includes a radioisotope which emits an alpha particle,
(b) providing the onco tool composition of claim 5 which includes a radioisotope which emits a beta particle,
(c) introducing both onco tool compositions into the patient.

27. A method for treating tumors in a patient, the method comprising
(a) providing the onco tool composition of claim 9 which includes a radioisotope which emits an alpha particle,
(b) providing the onco tool composition of claim 9 which includes a radioisotope which emits a beta particle,
(c) introducing both onco tool compositions into the patient.
